(19) United States
(12) Patent Application Publication
(10) Pub. No.: US 2003/0108883 A1
(43) Pub. Date: Jun. 12, 2003

(54) Methods for identifying compounds that inhibit or reduce PTP1B expression

(76) Inventors: Cristina M. Rondinone, Libertyville, IL (US); James M. Trevillyan, Grayslake, IL (US); Bradley A. Zinker, Vernon Hills, IL (US); Jeffrey F. Waring, Franklin, WI (US); Mike Jirousek, San Diego, CA (US); Madeline M. Butler, Santa Fe, CA (US); Lex M. Cowsert, Pittsburgh, PA (US); Brett P. Monia, Encinitas, CA (US); Jacqueline Wyatt, Encinitas, CA (US)

Correspondence Address: STEVEN F. WEINSTOCK; ABBOTT LABORATORIES, 100 ABBOTT PARK ROAD, DEPT. 377/AP6A, ABBOTT PARK, IL, 60064-6008, US

(21) Appl. No.: 10/074,194

(22) Filed: Feb. 12, 2002

Related U.S. Application Data
(60) Provisional application No. 60/268,399, filed on Feb. 13, 2001.

(51) Int. Cl. C12Q 1/68; G01N 33/53; G01N 33/542

(52) U.S. Cl. 435/6; 435/7.9

(57) ABSTRACT

The present invention relates to methods for identifying compounds that inhibit PTP1B mRNA and protein expression in insulin resistant non-human mammals.

### FIELD OF THE INVENTION

The present invention relates to biological markers for PTP1B inhibition or reduction. Specifically, the present invention relates to methods for measuring the downregulation of the p85α regulatory subunit of phosphotidylinositol-3-kinase (hereinafter “PI3-K”) and the upregulation of p55α and/or p50α isoforms in response to in vivo inhibition or reduction of protein-tyrosine phosphatase 1B (hereinafter “PTP1B”) in insulin resistant mammals. Moreover, the present invention relates to an in vivo marker for pharmacodynamic measurements and mechanism of action determinations of small molecule drugs which inhibit or reduce PTP 1B activity. Finally, the present invention also provides a method to screen agents for activity that down modulates p85α and upregulates PI3-kinase p85α isoforms as drugs for the treatment of type 2 diabetes.

### BACKGROUND OF THE INVENTION

Type 2 diabetes is a polygenic disease affecting over 100 million people worldwide. Affected patients manifest insulin resistance, hyperinsulinemia, and hyperglycemia (Kenner, et al., J. Biol. Chem., 271:19810-19816 (1996)). The molecular mechanism underlying the insulin resistance is not well understood but appears to involve a defect in the post-insulin receptor (IR) signal transduction pathway (Seely, et al., Diabetes, 45:1379-1285 (1996)). The IR is a receptor tyrosine kinase, and the binding of insulin to its receptor results in autophosphorylation of the IR and tyrosyl phosphorylation of IR substrate proteins (Calera, et al., J Biol. Chem., 275:6308-6312 (2000); Salmeen, et al., Mol. Cell, 6:1401-1412 (2000); Goldstein et al., J. Biol. Chem., 275:4283-4289 (2000); McGuire, et al., Diabetes, 40:939-942 (1991)). Protein tyrosine kinases and protein tyrosine phosphatases are important regulators of insulin signal transduction. Much attention has been focused on PTP1B, which inhibits insulin phosphorylation of the IR and insulin receptor substrates. Mice deficient in PTP1B expression have increased insulin sensitivity and low adiposity with resistance to weight gain on a high fat diet. In addition, the mice show increased basal metabolic rate and total energy expenditure (Ahmad, et al., Metabolism, 44:1175-1184 (1995); Elchebly, et al., Science, 283:1544-1548 (1999)). Thus, while it is clear that PTP 1B plays a role in insulin sensitivity and glucose homeostasis, there is a need in the art to understand how PTP1B acts in diabetes.

### SUMMARY OF THE INVENTION

The present invention relates to biological markers of PTP 1B inhibition or reduction. The markers of the present invention include the decrease or downregulation of the p85α regulatory subunit of PI3-kinase and the upregulation of p55α and/or p50α isoforms in response to the in vivo inhibition of PTP1B in insulin resistant non-human mammals. The biological markers of the present invention can be used as (1) a determinant for the in vivo activity of drugs which inhibit PTP1B; (2) a method for measuring the pharmacodynamics of PTP1B inhibitors; (3) a method for screening for novel agents for the treatment of insulin resistance and type 2 diabetes which increase insulin sensitivity by decreasing PI3-kinase p85α subunit and/or upregulating PI3-kinase p85α isoforms; and (4) an in vivo marker of increased insulin sensitivity.

More specifically, the present invention relates to a method for identifying a test compound that inhibits PTP 1B expression in liver or fat of a non-human mammal. The method involves the following steps. First, an insulin resistant non-human mammal, such as a mouse, rat, monkey, chimpanzee, dog or cow, is treated with a test compound for a time and under conditions sufficient to allow a change in the level of expression of at least one of PTP 1B mRNA and/or protein in the liver or fat of said mammal. Second, the fat and/or liver from said mammal is removed. Third, the levels of PI3-kinase p85α and p50α and/or p55α isoforms in the fat or liver are detected. Fourth, a determination is made whether or not the test compound inhibits the level of expression of at least one of PTP 1B mRNA and/or protein in the liver or fat based upon the levels of p85α and p50α and/or p55α isoforms detected in the fat or liver.

In another embodiment, the present invention relates to a method for identifying a test compound that downregulates PTP1B expression in liver or fat of a non-human mammal. The method involves the following steps. First, an insulin resistant non-human mammal, such as a mouse, rat, monkey, chimpanzee, dog or cow, is treated with a test compound for a time and under conditions sufficient to allow a change in the level of expression of at least one of PTP 1B mRNA or protein in the liver or fat of said mammal. Second, the fat and/or liver from said mammal is removed. Third, the levels of PI3-kinase p85α and p50α and/or p55α isoforms in the fat or liver are detected. Fourth, a determination is made whether or not the test compound downregulates the level of expression of at least one of PTP 1B mRNA or protein in the liver or fat based upon the levels of p85α and p50α and/or p55α isoforms detected in the fat or liver.

In yet a further embodiment, the present invention also relates to a method for identifying a test compound that increases insulin sensitivity and reduces blood glucose in an insulin resistant non-human mammal. The method involves the following steps. First, an insulin resistant non-human mammal, such as a mouse, rat, monkey, chimpanzee, dog or cow is treated with a test compound for a time and under conditions sufficient to allow for a change in the level of expression of at least one of PTP 1B mRNA or protein in the liver or fat of said mammal. Second, the fat or liver from said mammal is removed. Third, the levels of PI3-kinase p85α and p50α and/or p55α isoforms in the fat or liver are detected. Fourth, a determination is made whether or not the test compound increases insulin sensitivity and reduces blood glucose in the non-human mammal based upon the levels of p85α and p50α and/or p55α isoforms detected in the fat or liver.

In yet a further embodiment, the present invention also relates to a method for identifying a test compound that increases the levels of IRS-2 in the liver of an insulin resistant non-human mammal. The method involves the following steps. First, an insulin resistant non-human mammal, such as a mouse, rat, monkey, chimpanzee, dog or cow is treated with a test compound for a time and under conditions sufficient to allow for a change in the level of IRS-2 expression in the liver of said mammal. Second, the liver and fat from said mammal are removed. Third, the levels of IRS-2 in the liver are detected. Fourth, a determination is made whether or not the test compound increases the level of IRS-2 in the liver of the non-human.

In yet a further embodiment, the present invention relates to a method for identifying a test compound that downregulates the level of expression of at least one gene involved in lipogenesis. The method involves the following steps. First, an insulin resistant non-human mammal, such as a mouse, rat, monkey, chimpanzee, dog or cow, is treated with a test compound for a time and under conditions sufficient to allow for a downregulation in the level of expression of at least one gene involved in lipogenesis in a mammal. Second, the fat from said mammal is removed. Third, the level of expression of at least one gene involved in lipogenesis is determined. Fourth, a determination is made whether or not the test compound downregulates the level of expression of at least one gene involved in lipogenesis in the fat of the mammal.

In yet a further embodiment, the present invention relates to a method for identifying a test compound that downregulates the level of expression of at least one gene involved in gluconeogenesis. The method involves the following steps. First, an insulin resistant non-human mammal, such as a mouse, rat, monkey, chimpanzee, dog or cow, is treated with a test compound for a time and under conditions sufficient to allow for a downregulation in the level of expression of at least one gene involved in gluconeogenesis in a mammal. Second, the liver from said mammal is removed. Third, the level of expression of at least one gene involved in gluconeogenesis is determined. Fourth, a determination is made whether or not the test compound downregulates the level of expression of at least one gene involved in gluconeogenesis in the liver of the mammal.

### BRIEF DESCRIPTION OF THE FIGURES

FIG. 1 shows PTP1B protein expression levels in the liver and fit in diabetic, obese mice (ob/ob) and their lean (ob/+) littermates after treatment with various concentrations of a PTP1B antisense oligonucleotide.

FIG. 2 shows the treatment of diabetic, obese mice (ob/ob) and their lean (ob/+) littermates with various concentrations of PTP1B antisense oligonucleotide.

FIG. 3 shows PTP 1B protein levels in liver (FIG. 3A), fat (FIG. 3B), and skeletal muscle (FIG. 3C) from ob/ob mice treated for six weeks i.p. twice per week with PTP1B antisense (ANTISENSE) at the indicated dose. Skeletal muscle (FIG. 3C) was only tested at the 25 mg/kg dose for reduced protein level.

FIG. 4 shows non-fasting plasma glucose (FIG. 4A) and insulin (FIG. 4B) levels vs. time in PTP1B antisense treated ob/ob mice (25, 2.5, 0.25 mg/kg). Change in AUC for plasma glucose after an i.p. glucose tolerance test (FIG. 4C) and change in plasma glucose level after an insulin tolerance test (FIG. 4D) in ob/ob mice. Non-fasting plasma glucose (FIG. 4E) levels vs. time in PTP1B antisense treated db/db mice (50, 25, 10 and 50 mg/kg UC).

FIG. 5 shows representative immunoblots using anti-IRS-2 antibodies (FIG. 5A and FIG. 5B) or anti-p85α whole antiserum that recognized all p85 isoforms (panels C through F) were quantified.

FIG. 6A and FIG. 6B show IRS-2 protein levels in the liver and fat in saline control or PTP1B antisense-treated mice.

FIG. 7A shows that ob/ob mice treated with a PTP 1B antisense-oligonucleotide exhibited an increased basal, but mainly insulin-induced PKB phosphorylation without changing protein levels. FIG. 7B shows that PEPCK mRNA was decreased by 2.5 fold in ob/ob mice treated with a PTP 1B antisense-oligonucleotide.

FIG. 8 shows the effect of PTP1B reduction on body weights in ob/ob mice.

FIG. 9 shows the reduction of adiposity after PTP 1B antisense oligonucleotide treatment.

FIG. 10 shows the reduction of SREBP1 target genes, Spot 14 and FAS by PTP1B antisense oligonucleotide treatment.

FIG. 11 shows that PTP 1B reduction lowers triglyceride levels in epididymal fat.

FIG. 12 shows the effect of PTP1B reduction on insulin signaling.

FIG. 13 shows gene expression changes for some of the genes regulated by PTP1B ASO treatment in ob/ob adipose tissue. The change in gene expression is shown relative to saline-treated control mice. Genes that are shown in red were upregulated and genes that are shown in green were downregulated.

FIG. 14 shows Q-PCR results showing the regulation of adipsin (A) and PAI-1 (B) in adipocytes. The results are an average from 4 individual ob/ob mice treated with PTP1B ASO at the indicated dose levels.

FIG. 15 is a graph showing some of the gene expression changes in liver and muscle from ob/ob mice treated with PTP1B ASO. The results in liver are from mice treated at 50, 25 and 2.5 mg/kg and the results in muscle are from mice treated at 25 mg/kg.

FIG. 16 is a histopathology slide showing sections of liver from an ob/ob mouse treated with saline or with PTP1B ASO at 25 mg/kg. The top sections show 4× magnification, the bottom show 10× magnification.

FIG. 17 is a graph showing the change in gene expression of PEPCK, fructose-1,6-bisphosphatase and glucose-6-phosphatase in livers from ob/ob mice treated with PTP1B ASO. The change in gene expression is shown relative to saline-treated control mice.

### DETAILED DESCRIPTION OF THE INVENTION

Insulin resistant mammals possess a diminished ability to properly metabolize glucose. Such mammals typically respond poorly, if not at all, to insulin therapy. In insulin resistant mammals, tissues that exhibit resistance to insulin include the liver, fat and skeletal muscle. Moreover, these mammals contain increased levels of PTP1B in their tissue, particularly in the liver and fat. Unfortunately, insulin resistance causes or contributes to a number of medical disorders such as obesity, hypertension, atherosclerosis and the like. Eventually, insulin resistance can progress to a point where a diabetic state, namely, type 2 diabetes is reached.

The present invention provides assay methods for identifying compounds that are potentially useful as therapeutic agents for treating mammals that are insulin resistant. Additionally, the present invention also provides a method for improving the insulin sensitivity and glucose tolerance of an insulin resistant mammal by administering an effective amount of a compound that reduces the level of expression of at least one of PTP1B mRNA and/or protein in such a mammal. The present invention further relates to methods of treating an insulin resistant mammal with a compound identified by the methods of the present invention for the purpose of increasing insulin responsiveness and improving glucose tolerance in such a mammal.

In one embodiment, the present invention relates to a method for identifying a test compound that inhibits or downregulates (decreases) the level of expression of PTP1B mRNA and protein in a mammal. In this method, a compound can be identified as a potentially useful therapeutic agent based upon its ability to inhibit or downregulate the level of expression of at least one of PTP1B mRNA and/or protein expression in the tissue of an insulin resistant mammal. Preferably, the test compound inhibits or reduces the level of expression of PTP1B mRNA in the liver and PTP1B protein in the liver and/or fat in said mammal.

The method of the present invention is based upon the identification by the inventors of certain biological markers for PTP1B inhibition or reduction in insulin resistant mammals. These biological markers are derived from the regulatory subunit of PI3-kinase. PI3-kinase is a heterodimeric enzyme composed of a regulatory subunit (p85α and β) and 110 kD catalytic subunit. Several isoforms (also known as splice variants) of p85α art known in the art, namely, p50α and p55α (See, Y. Terauchi et al. Nat Genet., 21:230 (1999)). Additional isoforms of p85α, p55α and p50α, are also known in the art (See, K. Inukai et al., J Biol Chem., 272(12):7873 (1997)).

The inventors have found that examining the levels of expression of one or more of the regulatory subunits of PI3-kinase in the tissues of an insulin resistant mammal provides useful biological markers of PTP1B inhibition or reduction. Preferably, the level of expression of one or more of the regulatory subunits of PI3-kinase is examined in the fat or liver. More specifically, the inventors have discovered that a downregulation (or decrease) in p85α expression and an upregulation (or increase) of p50α and/or p55α isoform expression in an insulin resistant mammal is associated with an inhibition or downregulation in PTP1B mRNA and protein expression in such a mammal. The inventors have found that when insulin resistant mammals are administered compounds identified using the method of the present invention that these mammals exhibit increased insulin sensitivity and a reduction in blood glucose without the increased weight gain seen with treatment with compounds such as thiazolidinediones (TZD). Additionally, the inventors have also found that these compounds also decrease fat depots and triglyceride levels by downregulating genes involved in lipogenesis.

The method of the present invention involves administering to an insulin resistant non-human mammal a test compound for a time, amount and under conditions sufficient to allow for a change in the level of expression of at least one of PTP1B mRNA and/or protein in a mammal. Preferably, there is a decrease in the level of expression of at least one of PTP1B mRNA and/or protein. The test compound is administered to the mammal at any desired concentration. Typically, the test compound will be tested over a wide range of concentrations.

After a sufficient period of time has elapsed to allow for a change in the level of expression of at least one of PTP1B mRNA and/or protein, the liver and/or fat from the mammal is obtained using techniques known in the art. The levels of the regulatory subunits of PI3-kinase are then measured. Preferably, the levels of the p85α and/or p50α and p55α isoforms are determined using techniques known in the art for isolating and quantifying proteins, such as immunoblotting, column chromatography, gel filtration, etc. A test compound that downregulates p85α and upregulates the p50α and/or p55α isoforms indicates that the test compound possesses potential as a therapeutic agent.

In another embodiment, the present invention relates to a method for identifying a test compound that increases insulin sensitivity and reduces blood glucose in an insulin resistant mammal. The method of the present invention involves administering to an insulin resistant non-human mammal a test compound for a time, amount and under conditions sufficient to allow for a change in the level of expression at least one of PTP1B mRNA and/or protein in a mammal. The test compound is administered to the mammal at any desired concentration. Typically, the test compound will be tested over a wide range of concentrations.

After a sufficient period of time has elapsed to allow for a change in the level of PTP1B protein expression, the liver and/or fat from the mammal is obtained. The levels of the regulatory subunits of PI3-kinase are then measured. Preferably, the levels of the p85α and/or p50α and p55α isoforms are determined using techniques known in the art for isolating and quantifying proteins, such as immunoblotting, column chromatography, gel filtration, etc. A reduction in the level of the p85α and an increase in the level of the p50α and/or p55α isoforms of PI3-kinase indicates that that the test compound possesses potential as a therapeutic agent for increasing the insulin sensitivity and reducing the blood glucose in an insulin resistant mammal.

In another embodiment, the present invention relates to a method for identifying test compounds that increase the levels of expression of IRS-2 protein in the tissue of an insulin resistant mammal. Preferably, the test compound increases the level of expression of IRS-2 protein in the liver of such a mammal. In this method, a test compound can be identified as a potentially useful therapeutic agent based upon its ability to increase the level of expression of IRS-2 protein in the tissue, preferably the liver, of an insulin resistant mammal. The inventors have discovered that when insulin resistant mammals are administered compounds identified pursuant to the method of the present invention that these mammals exhibit increased insulin sensitivity and a reduction in blood glucose.

The method of the present invention involves administering to an insulin resistant non-human mammal a test compound for a time, amount and under conditions sufficient to allow for a change in the level of expression of IRS-2 protein in a mammal. The test compound is administered to the mammal at any desired concentration. Typically, the test compound will be tested over a wide range of concentrations.

After a sufficient period of time has elapsed to allow for a change in the level of expression of IRS-2 protein, the liver from the mammal is obtained. The level of expression of IRS-2 protein is determined using techniques known in the art for isolating and quantifying proteins, such as immunoblotting, column chromatography, gel filtration, etc. An increase in the level of expression of IRS-2 protein indicates that the test compound possesses potential as a therapeutic agent for increasing the insulin sensitivity and reducing the blood glucose in an insulin resistant mammal.

In yet a further embodiment, the present invention relates to a method for identifying test compounds that downregulate the level of expression at least one gene involved in lipogenesis. In this method, a test compound can be identified as a potentially useful therapeutic agent based upon its ability to decrease the level of expression at least one gene involved in lipogenesis, such as, but not limited to, genes which encode spot14, ATP citrate-lyase, fatty acid synthase, SteroylCoa desaturases, lipoprotein lipase and PPARλ.

The method of the present invention involves administering to an insulin resistant non-human mammal, a test compound for a time, amount and under conditions sufficient to allow for a downregulation in the level of expression of at least one gene involved in lipogenesis in a mammal. The test compound is administered to the mammal at any desired concentration. Typically, the test compound will be tested over a wide range of concentrations.

After a sufficient period of time has elapsed to allow for a downregulation in the level of expression of at least one gene involved in lipogenesis, fat from the mammal is obtained. The level of expression is determined using techniques in the art for quantifying gene expression, such as, microarray analysis or quantitative PCR. A decrease in the level of expression of at least one gene involved in lipogenesis indicates that the test compound possesses potential as a therapeutic agent for decreasing fat depots and triglyceride levels in insulin-resistant mammals.

In yet a further embodiment, the present invention relates to a method for identifying test compounds that downregulate the level of expression of at least one gene involved in gluconeogenesis. In this method, a test compound can be identified as a potentially useful therapeutic agent based upon its ability to decrease the level of expression at least one gene involved in gluconeogenesis, such as, but not limited to, genes which encode for PEPCK, fructose-1,6-bisphosphatase and glucose-6-phosphatase).

The method of the present invention involves administering to an insulin resistant non-human mammal, a test compound for a time, amount and under conditions sufficient to allow for a downregulation in the level of expression of at least one gene involved in gluconeogenesis in a mammal. The test compound is administered to the mammal at any desired concentration. Typically, the test compound will be tested over a wide range of concentrations.

After a sufficient period of time has elapsed to allow for a downregulation in the level of expression of at least one gene involved in gluconeogenesis, the liver from the mammal is obtained. The level of expression is determined using techniques in the art for quantifying gene expression, such as, microarray analysis or quantitative PCR. A decrease in the level of expression of at least one gene involved in gluconeogenesis indicates that the test compound possesses potential as a therapeutic agent for the treatment of type II diabetes.

Suitable test compounds that can be used in the methods of the present invention include any molecule, such as, but not limited to, proteins, oligopeptides, small organic molecules, polysaccharides, oligonucleotides (sense or antisense), polynucleotide (sense or antisense), etc. The test compound can encompass numerous chemical classes, though they are typically organic molecules. The test compound can be obtained for a wide variety of sources including libraries of synthetic or natural compounds. For example, many methods are known in the art for the random and directed synthesis of a wide variety of organic compounds and biomolecules, including expression of randomized oligonucleotides. Alternatively, libraries of natural compounds in the form of bacterial, fungal, plant and animal extracts can be used. Additionally, natural or synthetically produced libraries and compounds are readily modified through conventional chemical, physical and biochemical methods.

The non-human mammal used in the methods of the present invention can be a mouse, rat, monkey, chimpanzee, dog, cow and the like.

Test compounds identified pursuant to the methods of the present invention can be administered to a patient alone or in a pharmaceutical composition where it is mixed with suitable carriers or excipient(s). Suitable excipients include but are not limited to fillers such as sugars, including lactose, sucrose, mannitol, sorbitol, and the like, cellulose preparations such as, maize starch, wheat starch, rice starch, potato starch, gelatin, gum tragacanth, ethyl cellulose, hydroxypropylmethylcellulose, sodium carboxymethylcellulose, polyvinylpyrrolidone (PVP), and the like, as well as mixtures of any two or more. Optionally, disintegrating agents can be included, such as cross-linked polyvinyl pyrrolidone, agar, alginic acid or a salt thereof, such as sodium alginate and the like.

In addition to the excipients, the pharmaceutical composition can include one or more of the following, carrier proteins such as serum albumin, buffers, binding agents, sweeteners and other flavoring agents; coloring agents and polyethylene glycol.

Suitable routes of administration for the compound or pharmaceutical composition include, but are not limited to, oral, rectal, transdermal, vaginal, transmucosal or intestinal administration, parenteral delivery, including intramuscular, subcutaneous, intramedullary injections, as well as intrathecal, direct intraventricular, intravenous, intraperitoneal, intranasal, intraocular, and the like.

For oral administration, the pharmaceutical composition can be formulated as tablets, pills, capsules, dragees, liquids, gels, syrups, slurries, suspensions and the like. For administration by injection, the compound or the pharmaceutical composition can be formulated in an aqueous solution. Preferably, the aqueous solution is in a physiologically compatible buffer such as Hank's solution, ringer's solution or a physiological saline buffer.

The pharmaceutical composition of the present invention can be manufactured using techniques known in the art, such as, but not limited to, conventional mixing, dissolving, granulating, dragee-making, levigating, emulsifying, encapsulating, entrapping, lyosphilizing processes or the like.

The present invention also relates to methods of treating an insulin resistant mammal with the previously described compound or pharmaceutical composition for the purpose of increasing insulin responsiveness and improving glucose tolerance in such a mammal. The treatment method of the present invention involves administering to a mammal the compound or pharmaceutical composition in a therapeutically effective amount sufficient to increase insulin responsiveness and to improve glucose tolerance in such a mammal. The mammal to be treated is most preferably a human. Most preferably, the human is suffering from type 2 diabetes.

As used herein, the term “therapeutically effective amount” means an amount that produces the effects for which it is administered. The exact dose will be ascertainable by one skilled in the art. As known in the art, adjustments based on age, body weight, sex, diet, time of administration, drug interaction and severity of condition may be necessary and will be ascertainable with routine experimentation by those skilled in the art.

Suitable routes of administration for the compound or pharmaceutical composition include, but are not limited to, oral, rectal, transdermal, vaginal, transmucosal or intestinal administration, parenteral delivery, including intramuscular, subcutaneous, intramedullary injections, as well as intrathecal, direct intraventricular, intravenous, intraperitoneal, intranasal, intraocular, and the like.

Improvements or increases in insulin sensitivity and blood glucose in a mammal being treated pursuant to the above-described method can be detected and monitored using techniques and diagnostic tests known in the art. For example, improvements in blood glucose can be monitored using a standard glucose tolerance test. Other examples of tests used for the determination of an improvement in insulin sensitivity are the insulin tolerance test (ITT) and the euglycemic-hyperinsulinemic clamp. The GTT and ITT are performed by providing glucose or insulin intraperitoneally, respectively, to a rodent and measuring the blood glucose response over the ensuing 120 to 180 minutes. The clamp technique is completed by chronic catheterization of an artery (for sampling) and vein (for infusion of the glucose and insulin) and then infusing a constant rate of insulin with a variable infusion of glucose to maintain euglycemia. The glucose infusion rate can be used as an index of insulin sensitivity (the more glucose infused the more insulin sensitive the animal). Additionally, radioactive tracers can be infused with the insulin, which will permit measurements, along with the glucose infusion rate, of glucose production and disappearance, providing a more comprehensive picture of insulin sensitivity. All of the above techniques can be used with mammals, including humans.

An example of a compound identified pursuant to the methods of the present invention is ISIS-113715 (also referred to herein as “ASO”). ISIS-113715 is an antisense oligonucleotide that has the nucleotide sequence GCTCCTTCCACTGATCCTGC (SEQ. ID NO: 1). ISIS-113715 hybridizes to PTP1B mRNA at nucleotides 862-882 in the coding sequence. Specifically, ISIS-113715 downregulates the level of p85α expression and upregulates the level of p50α and p55α isoform expression in an insulin resistant mammal.

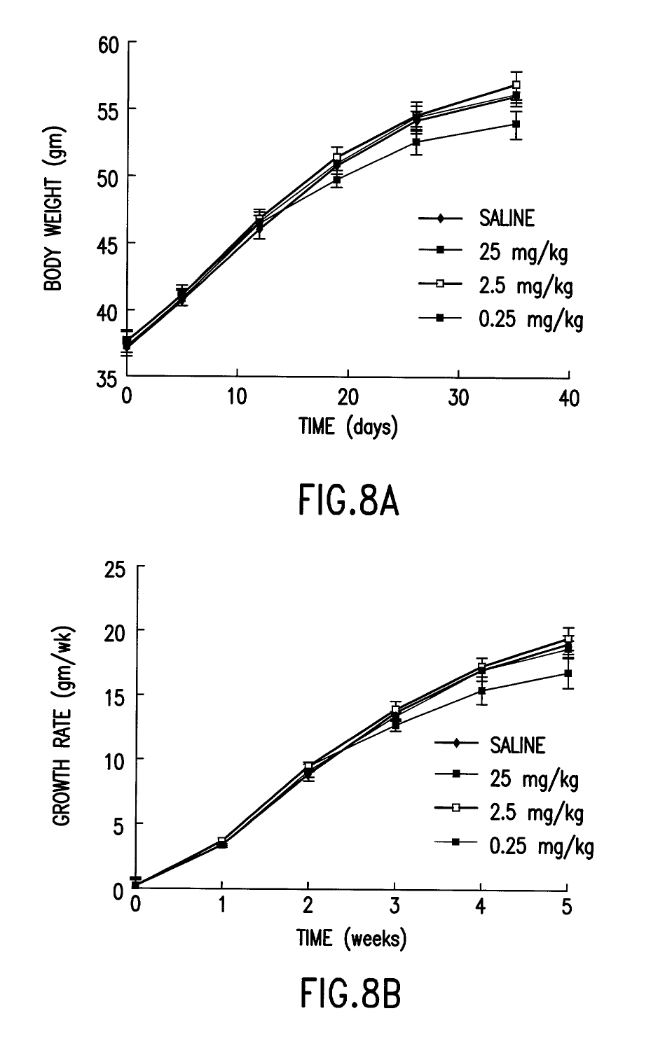

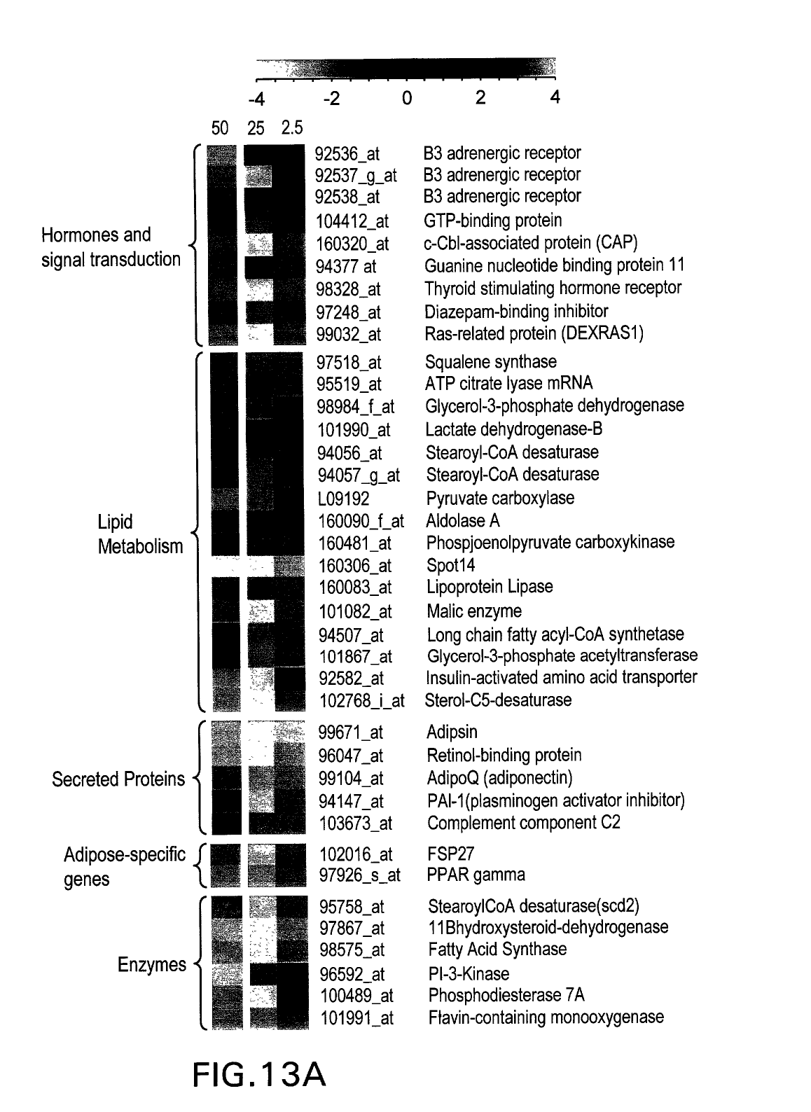

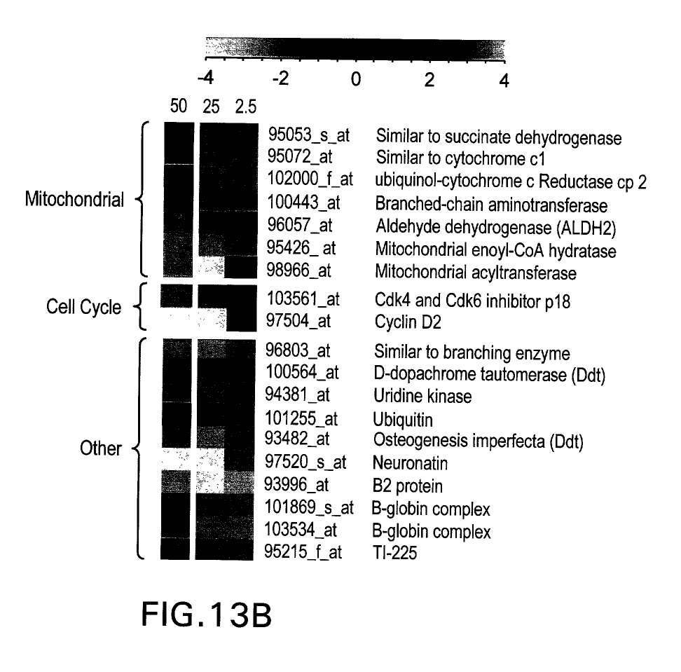

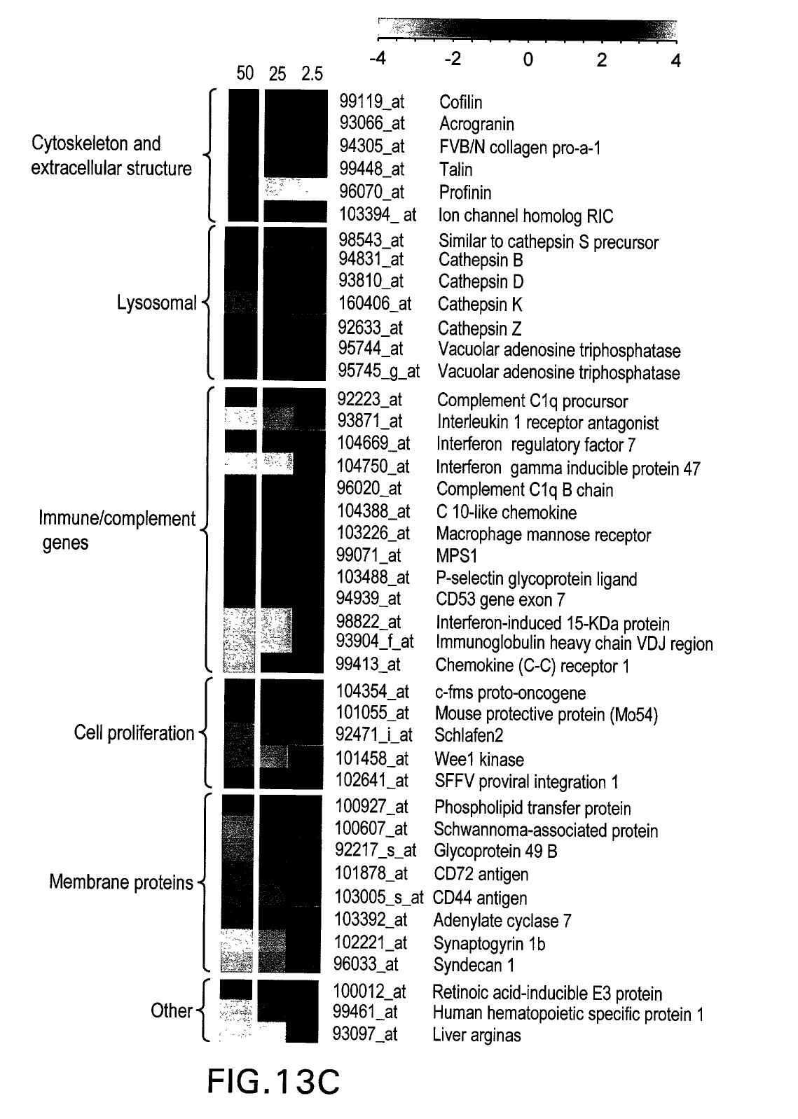

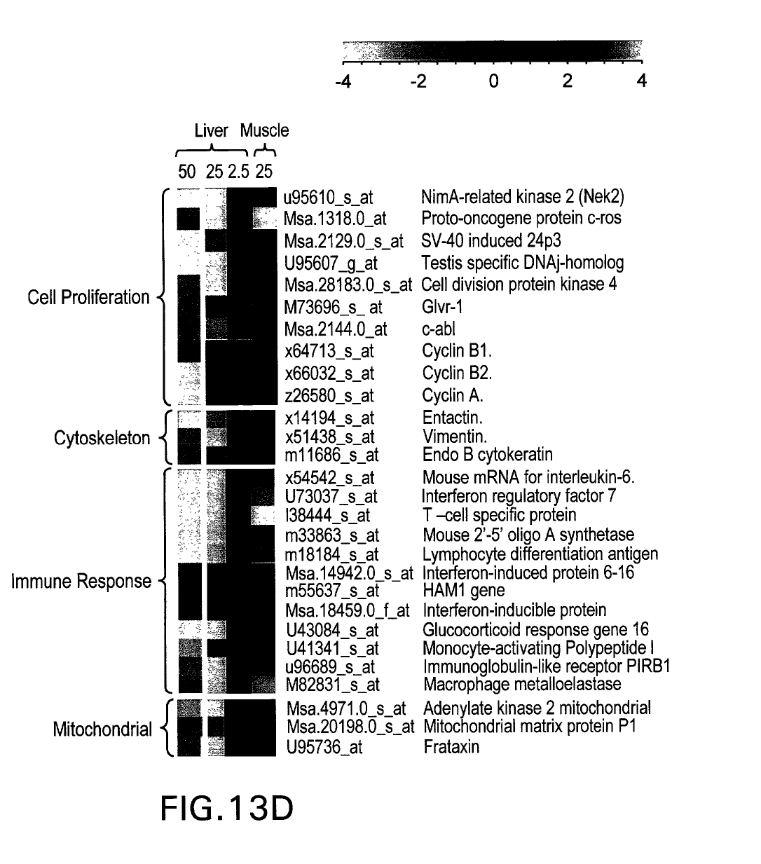

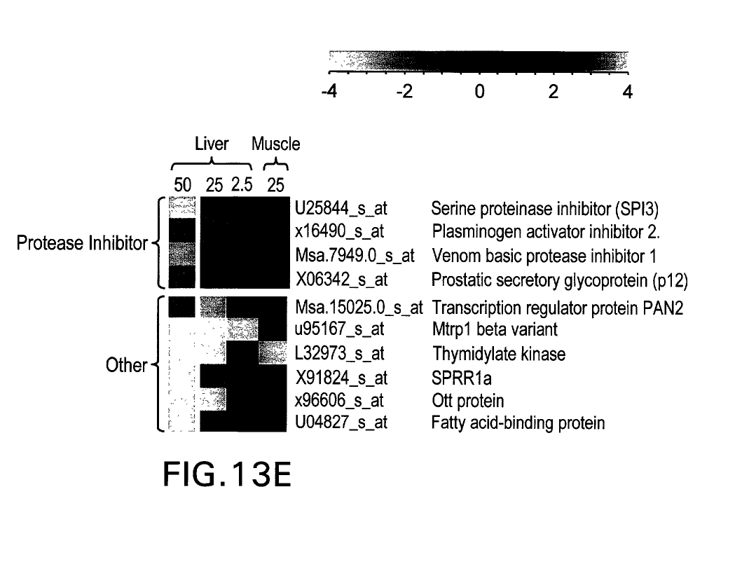

In addition, ISIS-113715 downregulates genes involved in lipogenesis and gluconeogenesis. Lipogenesis, the process of fatty acid synthesis, takes place in both adipose tissue and liver. Many of the genes involved in lipogenesis are regulated by the sterol regulatory element binding proteins (SREBP), including fatty acid synthase, glycerol-3-phosphate aceyltransferase, and spot 14 (Mater, et al., J. Biol. Chem., 274:32725-32732 (1999); Shimomura, et al., J. Biol. Chem., 273:35299-35306 (1998)). Other genes that are crucial for lipogenesis that may be directly or indirectly regulated by SREBP are stearoyl-CoA desaturase, pyruvate carboxylase, malic enzyme and long chain acyl-CoA synthetase. All of these genes are downregulated in adipose tissue treated with ISIS-113715 (See FIGS. 8 and 13). The inventors have found that the downregulation of the genes involved in lipogenesis decreases fat depots and triglyceride levels. Moreover, the inventors have also discovered that the genes in the liver involved in gluconeogenesis (such as, but not limited to, PEPCK, fructose-1,6-bisphosphatase and glucose-6-phosphatase) are also downregulated when treated with ISIS-113715.

ISIS-113715 can be administered to an insulin resistant mammal for the purpose of improving insulin sensitivity and reducing blood glucose without leading to increased weight gain as seen with TZD treatment.

By way of example and not of limitation, examples of the present invention will now be given.

### EXAMPLE 1

Rapid throughput screens for identifying antisense inhibitors against PTP1B were performed with 20-base chimeric antisense oligonucleotides (ANTISENSEs) where the first five bases and last five bases have a 2′-O-(2-methoxy)-ethyl (2′ MOE) modification. The 2′MOE modification increases binding affinity to complementary RNA sequences and increase resistance to nucleases, Dean, N. M., et. al. Pharmacology of 2′-O-(2-methoxy) ethyl modified antisense oligonucleotides, in Antisense Technology: Principles, Strategies and Applications, S. T. Crooke, Editor. Marcel Dekker. In press. The antisense oligonucleotides have a phosphorothiorate backbone and use an RNase H dependent mechanism for activity. Initial screens were conducted against rat PTP1B and ten antisense oligonucleotides were identified as hits, all of which targeted the same binding site within the coding region of the PTP1B mRNA. Subsequently, a series of in vitro characterization experiments were performed in primary rat and mouse hepatocytes, in which ISIS-113715 was consistently identified to be the most potent and specific oligonucleotide in reducing PTP1B mRNA levels. ISIS-113715 hybridizes to PTP1B mRNA at nucleotides 862882 in the coding sequence. The ISIS-113715 binding site is conserved across mouse, rat, monkey, and man.

### EXAMPLE 2

A six week antisense study in ob/ob mice using a dose range of 25, 2.5, 0.25, or 0 mg/kg, by i.p. administration two times a week, was performed. Additionally, separate groups of ob/ob mice were treated for 3 wk at 50 or 0 mg/kg, by i.p. administration two times a week. ob/ob mice and their lean littermates (obl+) of 6-7 weeks of age (Jackson Laboratories, Bar Harbor, Me.) were acclimated to the animal research facilities for ˜5 days. The following investigations were conducted in accordance with each institution's IACUC guidelines. Animals were housed and maintained on mouse chow (ob/ob Labdiets #5015, St. Louis, Mo.) ad libitum. After acclimation the ob/ob and lean mice were weighed and tail snip plasma glucose levels were determined by the glucose oxidase method (Precision G glucose meter, Abbott Laboratories, North Chicago, Ill.). The animals were randomized based on glucose levels and body weight. Baseline plasma insulin samples were also taken from a subset of the animals representing each treatment group once randomized (n—10 ob/ob and n=10 lean littermates; ELISA, ALPCO Diagnostics, Windham, N.H.). Treatment groups were: 1) ob/ob PTP1B antisense 25 mg/kg, 2.5, 0.25 and saline (n=10/treatment) for 6 wk and 50 mg/kg (n=9) for 3 wk; lean littermates PTP1B antisense 25 mg/kg, 2.5, 0.25 and saline (n=10/treatment) for 6 wk, and universal control (UC) for 4 wk (n=8/treatment glucose and n=3/treatment for all other parameters). All mice were dosed two times a week (ob/ob) i.p. The antisense oligonucleotides were weighed and resuspended in saline at a concentration of 25 mg/ml. The suspension was vortexed well and allowed to sit at room temperature for 15 min, following which it was filtered through a syringe filter (0.2 μm; Gelman Acrodisc), 2 μl of the filtrate was diluted in 1 ml of H₂O and OD read at 260. The formula used to calculate the concentration was as follows: (OD*dilution factor*molecular weight)/(extinction coefficient*1000)=concentration in mg/ml. The stock was diluted to the desired concentration for injection in sterile saline and frozen at 20° C. For subsequent use, the stock was thawed, heated to 37° C. and vortexed well before using. At the and of each week tail bleed glucose and insulin levels as well as body weight were determined under non-fasting conditions by 10 am. After 3 or 6 week of treatment body weight was measured and final tail bleed samples and a cardiac puncture were performed (after dry ice asphyxiation) for measurement of non-fasting glucose, and insulin (ob/ob only). Animal tissues (liver, abdominal fat, and skeletal muscle (ob/ob only) were harvested and frozen immediately in liquid nitrogen for further analyses.

### EXAMPLE 3

Tissues were sonicated (using a Branson 450 Sonifier) in lysis buffer containing 20 mM Tris-HCl (pH 7.4), 1% Triton X-100, 10% glycerol, 150 mM NaCl, 2 mM EDTA, 25 mM β-glycerophosphate, 20 mM sodium fluoride, 1 mM sodium orthovanadate, 2 mM sodium pyrophosphate, 10 μg/ml leupeptin, 1 mM benzamidine, 1 mM 4-(2-aminoethyl) benzenesulfonyl fluoride hydrochloride (Calbiochem), 1 mM microcystin and rocked for 40 min at 4° C. Detergent-insoluble material was sedimented by centrifugation at 12,000 x g for 10 min at 4° C. Cell lysate proteins (50 μg of protein) were separated by SDS/PAGE on 10% and 7.5% gels. Proteins were transferred from the gel to nitrocellulose sheers and blocked in 5% milk. The blots were probed with various primary antibodies as follows: anti-PTP1B, anti-IRS-1 (PH domain), anti-IRS-2, anti-p85 (whole antiserum) antibodies (Upstate Biotechnology, Lake Placid, N.Y.), anti-IRβ antibody (Transduction Laboratories, San Diego, Calif.), phospho-protein kinase B (PKB) antibody (New England Biolabs, Beverly, Mass.) according to the recommendations of the manufacturer. The proteins were detected by enhanced chemiluminescence with horseradish peroxidase-labeled secondary antibodies (Amersham). The intensity of the bands was quantitated with a laser densitometer (Molecular Dynamics, Sunnyvale, Calif.).

### EXAMPLE 4

RNA preparation was done by grinding approximately 100 mg of liver tissue in 1 ml of TRIzol reagent and analysis was done according to the Affymetrix Inc. protocol. Briefly, the RNA from four mice in PTP1B antisense-treated or control groups were pooled using equal amounts to make a total of 20 μg of RNA. cRNA was prepared using the Superscript Choice. system from Gibco BRL Life Technologies (Cat. No. 18090-019). The protocol was followed with the exception that the primer used for the reverse transcription reaction was a modified T7 primer with 24 thymidines at the 5′ end. The sequence was:5′-GGCCAGTGAATTGTAATACGACTCACTATAGGGAGGCGG-(dT)₂₄-3′ (SEQ ID NO:2).

Following this, labeled cRNA was synthesized from the cDNA using the Enzo RNA Transcript Labeling Kit (Cat. No. 900182) according to the manufacturers instructions. Approximately 20 μg of cRNA was then fragmented in a solution of 40 mM Tris-acetate, pH 8.1, 100 mM KOAc, and 30 mM MgOAc at 94° C. for 35 minutes.

Labeled cRNA was hybridized to the Affymetrix GeneChip Test2 Array to verify the quality of labeled cRNA. Following this, cRNA was hybridized to the Affymetrix MU11K A and B chip. The cRNA was hybridized overnight at 45° C. The data was analyzed using Affymetrix GeneChip Version 3.2 software and Spotfire.Net Version 5.0. The microarray experiment was repeated using RNA isolated a second time from the same mouse livers. The results are an average of the two experiments.

### EXAMPLE 5

ob/ob and db/db mice and their lean (ob/+) littermates of 6-7 weeks of age (Jackson Laboratories, Bar Harbor, Me.) were acclimated to the animal research facilities for ˜5 days. The following investigations were conducted in accordance with each institution IACUC guidelines. Animals were housed (5 per cage, ob/ob, C57BL/6J-Lep^{ob}; 4 per cage, db/db, C57BLKS/J-m+/+Lepr^{db}; 2 per cage least littermates) and maintained on mouse chow (ob/ob Labdiets #5015, St. Louis, Mo.; db/db Harlan-Teklad rodent diet #8604 Madison, Wis.; 26% fat calories) ad libitum. After acclimation the ob/ob and lean mice were weighed and tail snip plasma glucose levels were determined by the glucose oxidase method (Precision G glucose meter, Abbott Laboratories, North Chicago, Ill.). The animals were randomized based on glucose levels and body weight. Baseline plasma insulin samples were also taken from a subset of the animals representing each treatment group once randomized (n=10 ob/ob and n=10 lean littermates; ELISA, ALPCO Diagnostics, Windham, N.H.). Treatment groups were: 1) ob/ob PTP1B antisense 25 mg/kg, 2.5, 0.25 and saline (n=10/treatment) for 6 wk and 50 mg/kg (n=9) for 3 wk; lean littermates PTP1B antisense 25 mg/kg, 2.5, 0.25 and saline (n=10/treatment) for 6 wk, and 2) db/db PTP1B antisense 50 mg/kg, 25, 10, saline, and universal control (UC) for 4 wk (n=8/treatment glucose and n=3/treatment for all other parameters). All mice were dosed twice/wk (ob/ob) and once/wk (db/db) i.p. The antisense oligonucleotides were weighed and resuspended in saline at a concentration of 25 mg/ml. The suspension was vortexed well and allowed to sit at room temperature for 15 min, following which it was filtered through a syringe filter (0.2 μm; Gelman Acrodisc). 2 μl of the filtrate was diluted in 1 ml of H₂O and OD read at 260. The formula used to calculate the concentration was as follows: (OD*dilution factor*molecular weight)/(extinction coefficient*1000)=concentration in mg/ml. The stock was diluted to the desired concentration for injection in sterile saline and frozen at 20° C. For subsequent use, the stock was thawed, heated to 37° C. and vortexed well before using. At the end of each week tail bleed glucose and insulin (ob/ob only) levels as well as body weight were determined under non-fasting conditions by 10 am. A gross estimation of food consumption (ob/ob only) each wk was determined as follows; food was measured at 10 am at the start and end of a 24 hr period (same 24 hr period each wk), the weight was subtracted from the previous day and divided by the number of mice per cage for an index of estimated 24 hr food consumption.

### EXAMPLE 6

A UC oligonucleotide pool (ISIS 29848) was synthesized as a mixture of A (adenine), G (guanosine), T (thymine) and C (Cytosine) basis so that the resulting preparation contains an equimolar mixture of all possible 4¹⁹ oligonucleotides. The oligonucleotide chemistry of ISIS 29848 is identical to that of ISIS-113715.

### EXAMPLE 7

An i. p. GTT (ob/ob only) was performed at 0.5 gm/kg (50% solution of D-50 Dextrose, Abbott Laboratories, North Chicago, Ill.). After a 3 hr fast beginning at ˜6:30 AM and after a baseline 0 min sample an i.p. injection of glucose was given and tail bleed glucose samples were additionally taken at 15, 30, 60 and 120 min. Animals were studied after 3 or 6 wk of treatment. The 6 wk 25 mg/kg and saline control treatments underwent an i.p. ITT (ob/ob only) at 2 U insulin/kg (50% solution in 0.1% BSA (R-Insulin, E. Lilly and Co., Indianapolis, Ind.)). After an overnight fast and a baseline 0 min tail bleed glucose sample an i.p. injection of insulin was given and additional glucose samples were taken at 15, 30, 60 and 120 min.

### EXAMPLE 8

Body weights were not different over the duration of the study between saline control and ANTISENSE treatment at 0.25 or 2.5 mg/kg. Body weights were not different at the end of week 5 between 25 mg/kg antisense oligonucleotide and saline control (p=0.12). Week 6 body weights, following an overnight fast, were 54.7±0.8 vs. 53.5±0.9 gm, saline control vs. 25 mg/kg antisense oligonucleotide (p>0.05). Weekly weight gain was not different between treatments, with borderline significance with the highest dose antisense oligonucleotide treatment at 6 week (p=0.14). Epididymal fat weight (4.9±0.1, 3.0±0.2, 4.2±0.3, 5.0±0.3 gm; saline, 25, 2.5, 0.25 mg/kg antisense oligonucleotide) was reduced 42% with high dose treatment (p<0.05, normalized to brain weight) and unchanged with lower dose treatments vs. saline control. Liver weight (4.0±0.1, 5.6±0.3, 4.5±0.2, 3.6±0.2 gm; saline, 25, 2.5, 0.25 mg/kg antisense oligonucleotide) was increased 36% with 25 mg/kg antisense oligonucleotide treatment only (p<0.05, normalized to brain weight). It is possible that metabolism of lipids was altered such that flux through utilization pathways (and/or diminished storage) was enhanced resulting in reduced fat weight. Future studies are needed to determine the meaning and mechanism of this observation in this model. Estimated food consumption was decreased in the 25 mg/kg antisense oligonucleotide treated ob/ob mice compared to the saline controls during the 6^{th} week (P=0.03), and narrowly missed significance during the 4^{th} (P=0.08, 1-tail) and 3^{rd} weeks (P=0.05, 1-tail). There were no differences in food intake during weeks 1, 2 and 5 (p>0.05). The reduction in weight gain and food intake clearly cannot be responsible far the early normalization of glucose and decrease in insulin levels. Histopathology, blood chemistry, and molecular toxicology examination of the ob/ob mice in these studies did not reveal any adverse reaction to treatment with ISIS-113715 at pharmacologically relevant doses. Three-week exposure at the highest dose (50 mg/kg) produced modest elevations in serum transaminse levels above control levels; non-sequence specific elevations have been reported for other antisense molecules and this mouse model has normally high background levels reflective of the fatty morphology. A morphological change from a fatty liver phenotype in controls to a leaner phenotype in treated animals was observed along with changes in nuclear morphology within hepatocytes. Data from gene expression analysis using microarrays did not show evidence for any repair or proliferative response, hence the morphological changes were interpreted to be a direct result of the intended pharmacological activity.

### EXAMPLE 9

Rapid throughput screens for identifying ASO inhibitors selective against PTP1B were performed with 20-base chimeric ASOs where the first five bases and last five bases have a 2′-O-(2-methoxy)-ethyl (2′MOE) modification. The 2′MOE modification increases binding affinity to complementary RNA sequences and increase resistance to nucleases. The ASO oligonucleotides have a phosphorothiorate backbone and use an RNase H dependent mechanism for activity. Initial screens were conducted against rat PTP1B and ten ASOs were identified as hits, all of which targeted the same binding site within the coding region of the PTP1B mRNA. Subsequently, a series of in vitro characterization experiments were performed in primary rat and mouse hepatocytes, in which ISIS-113715 was consistently identified to be the most potent and specific oligonucleotide in reducing PTPlB MRNA levels. ob/ob of 6-7 weeks of age (Jackson Laboratories, Bar Harbor, Me.) were acclimated to the animal research facilities for 5 days. The following investigations were conducted in accordance with each institution IACUC guidelines. Animals were housed and maintained on mouse chow (ob/ob Labdiets #5015, St. Louis, Mo.;) ad libitum.

After acclimation, the ob/ob were weighed and tail snip glucose levels were determined by the glucose oxidase method (Precision G glucose meter, Abbott Laboratories, North Chicago, Ill.). The animals were randomized to the various treatment groups based on plasma glucose levels and body weight. Baseline plasma insulin samples were taken from a subset of the animals representing each treatment group once randomized (n=10 ob/ob and n=10 lean littermates; ELISA, ALPCO Diagnostics, Windham, N.H.). Treatment groups were: 1) ob/ob PTP1B ASO 25 mg/kg, 2.5, 0.25 and saline (n=10/treatment) for 6 wk). All mice were dosed i.p. twice/wk. At the end of each week tail bleed glucose and insulin (ob/ob only) levels as well as body weight were determined under non-fasting conditions by 10 am (as described above). At the end of the studies epididymal fat pads were frozen immediately in liquid nitrogen for further analysis.

Insulin (2U/kg in 0.1% BSA) or saline control was given i.p. after an overnight fast. Tissue samples from liver (0, 1, 5 min) were taken under both saline and insulin stimulated conditions (n=4/treatment/time point). Within each challenge (saline and insulin) were subgroups of saline or antisense treated (25 mg/kg) mice.

Tissues were sonicated (using a Branson 450 Sonifier) in lysis buffer containing 20 mM Tris-HCl (pH 7.4), 1% Triton X-100, 10% glycerol, 150 mM NaCl, 2 mM EDTA, 25 mM β-glycerophosphate, 20 mM sodium flouride, 1 mM sodium orthovanadate, 2 mM sodium pyrophosphate, 10 μg/ml leupeptin, 1 mM benzamidine, 1 mM 4-(2-aminoethyl) benzenesulfonyl fluoride hydrochloride, 1 mM microcystin and rocked for 40 min at 4° C. Detergent-insoluble material was sedimented by centrifugation at 12,000× g for 10 min at 4° C. Cell lysate proteins (50 μg of protein) were separated by SDS/PAGE on 10% and 7.5% gels. Proteins were transferred from the gel to nitrocellulose sheets and blocked in 5% milk. The blots were probed with various primary antibodies as follows: anti-PTP1B, anti-IRS-1 (PH domain), anti-IRS-2, anti-p85 (whole antiserum) antibodies (Upstate Biotechnology, Lake Placid, N.Y.), anti-IRβ antibody (Transduction Laboratories, San Diego, Calif.), phospho-protein kinase B (PKB) antibody (New England Biolabs, Beverly, Mass.) according to the recommendations of the manufacturer. The proteins were detected by enhanced chemiluminescence with horseradish peroxidase-labeled secondary antibodies (Amersham). The intensity of the bands was quantitated with a laser densitometer (Molecular Dynamics, Sunnyvale, Calif.).

RNA preparation was done by grinding approximately 100 mg of liver tissue in 1 ml of TRIzol reagent and analysis was done according to the Affymetrix Inc. protocol. Briefly, the RNA from four mice in PTP1B ASO-treated or control groups was pooled using equal amounts to make a total of 20 μg of RNA. cRNA was prepared using the Superscript Choice system from Gibco BRL Life Technologies (Cat. No.18090-019). The protocol was followed with the exception that the primer used for the reverse transcription reaction was a modified T7 primer with 24 thymidines at the 5′ end. The sequence was: 5′GGCCAGTGAATTGTAATACGACTCACTATAGGGAGGCGG-(dT)24-3′ (SEQ ID NO:2). Following this, labeled cRNA was synthesized according to the manufacturers instructions from the cDNA using the Enzo RNA Transcript Labeling Kit (Cat. No. 900182). Approximately 20 μg of cRNA was then fragmented in a solution of 40 mM Tris-acetate, pH 8.1, 100 mM KOAc, and 30 mM MgOAc at 94° C. for 35 minutes. Labeled cRNA was hybridized to the Affymetrix GeneChip Test2 Array to verify the quality of labeled CRNA. Following this, cRNA was hybridized to the Affymetrix MUl 1K A and B chip. The cRNA was hybridized overnight at 45° C. The data was analyzed using Affymetrix GeneChip Version 3.2 software and Spotfire.Net Version 5.0 The results are an average of the two experiments.

Total RNA was isolated and amplified as described. In vitro transcription was performed using MEGAscrip™ T7 Kit (Ambion) according to the manufactures protocol. Amplified antisense RNA was purified with Rneasy mini-kit and protocol (Qiagen) then eluted in a volume of 60 ul Rnase-Free water. The aRNA was quantified by fluorescenceusing Ribogreen dye (Molecular Probes) and the integrity of sample was assessed by separation on an Agilent 2100 Bioanalyzer, a high resolution electrophoresis system (AgilentTechnologies, Palo Alto, Calif.). RNA was fluorescently labeled and hybridized to a Mouse Gem 2 microarray. After scanning and data extraction, the results were exported to the GEMTools database for further gene expression analysis.

Real time PCR was performed using the Taqman® EZ RT-PCR Core Reagents kit (Perkin Elmer Part Number N808-0236). For the analysis, 100 ng of total RNA was used. The reactions were done in triplicate. The probe sequences for FAS mRNA were:

(SEQ. ID NO:3)

TGCATGACAGCATCCAAGACA-Forward Probe;

(SEQ. ID NO:4)

CTCTTCCCATGAGATTGGTACCA-Reverse Probe; and

(SEQ ID NO:5)

AGCTGAGCAGAAAGTCCAGCTGCTCCT-Taqman Probe.

The probe sequences for Spot14 mRNA were:

(SEQ ID NO:6)

CCCAGTTCCACCTGCACTTCT-Forward Probe;

(SEQ ID NO:7)

CTCCTGTGCTTTCCGGGTC-Reverse Probe; and

(SEQ ID NO:8)

CAGCCTCCATCACATCCTTACCCACC-Taqman Probe.

Statistical evaluation was performed via 1-way ANOVA and t-tests where appropriate using InStat (GraphPad Software, Inc., San Diego, Calif.). The level of significance was P<0.05 (two-sided test).

### EXAMPLE 10

Rapid throughput screens for identifying ASO inhibitors selective against PTP1B were performed with 20-base chimeric ASOs where the first five bases and last five bases have a 2′-O-(2-methoxy)-ethyl (2′MOE) modification. The 2′MOE modification increases binding affinity to complementary RNA sequences and increase resistance to nucleases. The ASO oligonucleotides have a phosphorothiorate backbone and use an RNase H dependent mechanism for activity. Initial screens were conducted against rat PTP1B and ten ASOs were identified as hits, all of which targeted the same binding site within the coding region of the PTP1B mRNA. Subsequently, a series of in vitro characterization experiments were performed in primary rat and mouse hepatocytes, in which ISIS-113715 was consistently identified to be the most potent and specific oligonucleotide in reducing PTP1B mRNA levels.

ob/ob mice and their lean littermates of 6-7 weeks of age (Jackson Laboratories, Bar Harbor, Me.) were acclimated to the animal research facilities for 5 days. The following investigations were conducted in accordance with each institution IACUC guidelines. Animals were housed (5 per cage, ob/ob, C57BL/6J-Lepob; 4 per cage, db/db, C57BLKS/J-m+/+Leprdb; 2 per cage lean littermates) and maintained on mouse chow (ob/ob Labdiets #5015, St. Louis, Mo.; db/db Harlan-Teklad rodent diet #8604 Madison, Wis.; 26% fat calories) ad libitum. After acclimation the ob/ob and lean mice were weighed and tail snip glucose levels were determined by the glucose oxidase method (Precision G glucose meter, Abbott Laboratories, North Chicago, Ill.). The animals were randomized to the various treatment groups based on plasma glucose levels and body weight. Baseline plasma insulin samples were taken from a subset of the animals representing each treatment group once randomized (n=10 ob/ob and n=10 lean littermates; ELISA, ALPCO Diagnostics, Windham, N.H.). Treatment groups were: 1) ob/ob PTP1B ASO 25 mg/kg, 2.5, 0.25 and saline (n=10/treatment) for 6 wk and 50 mg/kg (n=9) for 3 wk. All mice were dosed i.p.twice/wk. At the end of each week tail bleed glucose and insulin (ob/ob only) levels as well as body weight were determined under non-fasting conditions by 10 am (as described above). A gross estimation of food consumption was determined in ob/ob mice each wk as follows; food was measured at 10 am at the start and end of a 24 hr period (same 24 hr period each wk), the weight was subtracted from the previous day and divided by the number of mice per cage for an index of estimated 24 hr food consumption. At the end of the studies, liver, epididimal fat pads and skeletal muscle were harvested and frozen immediately in liquid nitrogen for further analysis.

Three saline control and three PTP1B ASO treated mice were utilized for histopathologic examination. Sections of liver, brain, lung, spleen, pancreas, myocardium, skeletal muscle, sciatic nerve, eye, kidney, and bone marrow were harvested at necropsy and fixed in 10% neutral buffered formalin for 24-48 hours. The specimens were then dehydrated through graded alcohols, and embedded in paraffin wax. Five micron sections were cut and stained with hematoxylin and eosin.

RNA preparation was done by grinding approximately 100 mg of liver tissue in 1 ml of TRIzol reagent and analysis was done according to the Affymetrix Inc. protocol (See, Wodicka L, et al., Nat Biotechnol., 15(13):1359-67 (1997)). Briefly, the RNA from four mice in PTP1B ASO-treated or control groups was pooled using equal amounts to make a total of 20 μg of RNA. cRNA was prepared using the Superscript Choice system from Gibco BRL Life Technologies (Cat. No.18090-019). The protocol was followed with the exception that the primer used for the reverse transcription reaction was a modified T7 primer with 24 thymidines at the 5′ end. The sequence was: 5′GGCCAGTGAATTGTAATACGACTCACTATAGGGAGGCGG-(dT)₂₄-3′ (SEQ ID NO:2). Following this, labeled cRNA was synthesized according to the manufacturer's instructions from the cDNA using the Enzo RNA Transcript Labeling Kit (Cat. No. 900182). Approximately 20 μg of cRNA was then fragmented in a solution of 40 mM Tris-acetate, pH 8.1, 100 mM KOAc, and 30 mM MgOAc at 94° C. for 35 minutes.

Labeled cRNA was hybridized to the Affymetrix GeneChip Test2 Array to verify the quality of labeled cRNA. Following this, cRNA was hybridized to the Affymetrix MU11K A and B chip for all treatments of muscle and liver and for fat treated at 25 mg/kg. The microarray experiment was repeated for the liver 50 mg/kg treatment group using RNA isolated a second time from the same mouse livers, and the results are an average of the two experiments. The MU-U74 V.2 chip was used for fat treated at 2.5 and 50 mg/kg, and the 25 mg/kg treatment done on the MU11K A and B was repeated on the MU-U74 V.2 chip. The CRNA was hybridized overnight at 45° C. The data was analyzed using Affymetrix GeneChip Version 3.2 software and Spotfire.Net Version 5.0.

Real time PCR was performed using the Taqman® EZ RT-PCR Core Reagents kit (Perkin Elmer Part Number N808-0236). For the analysis, 100 ng of total RNA was used. The reactions were done in triplicate.

The probe sequences for alipsin were:

(SEQ ID NO:9)

GCAGTCGAAGGTGTGGTTACGT-Forward Probe;

(SEQ ID NO:10)

CTCGCGTCTGTGGCAATGGCAA-Taqman Probe; and

(SEQ ID NO:11)

GGGTATAGACGCCCGGCTT-Reverse Probe.
The probe sequences for PAI-1 were:

(SEQ ID NO:12)

CTCCACAGCCTTTGTCATCTCA-Forward Probe;

(SEQ ID NO:13)

CATGGCCCCCACGGAGATGGTT-Taqman Probe; and

(SEQ ID NO:14)

GTGCCGAACCACAAAGAGAAA-Reverse Probe.

### EXAMPLE 11

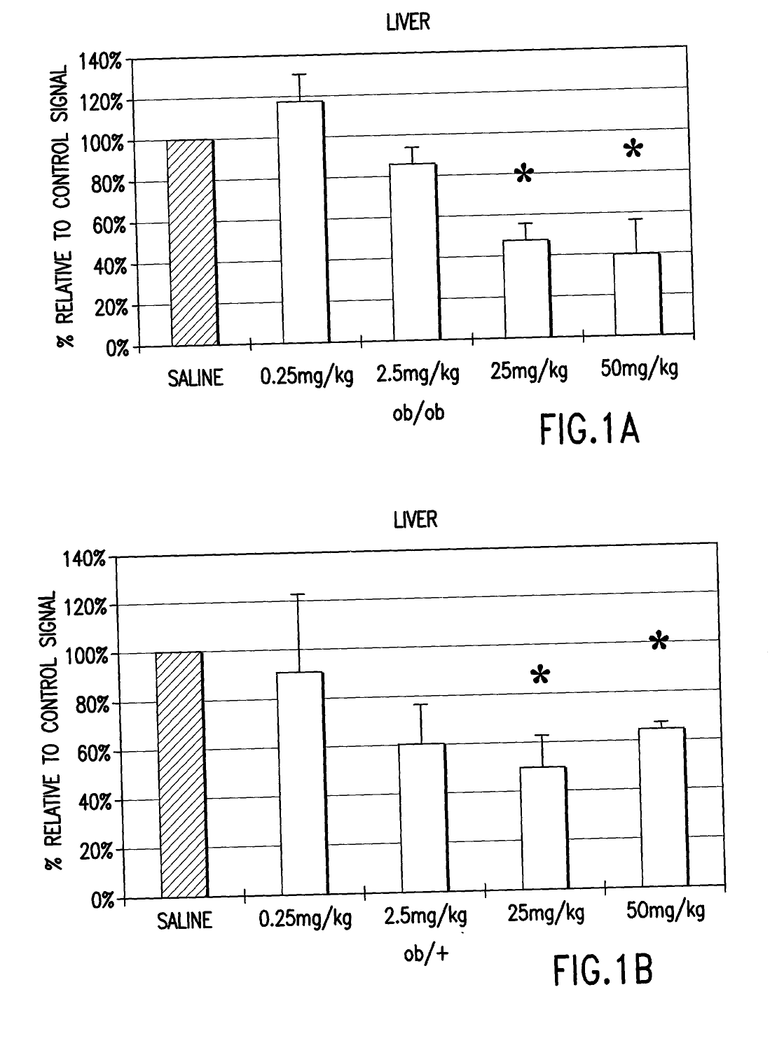

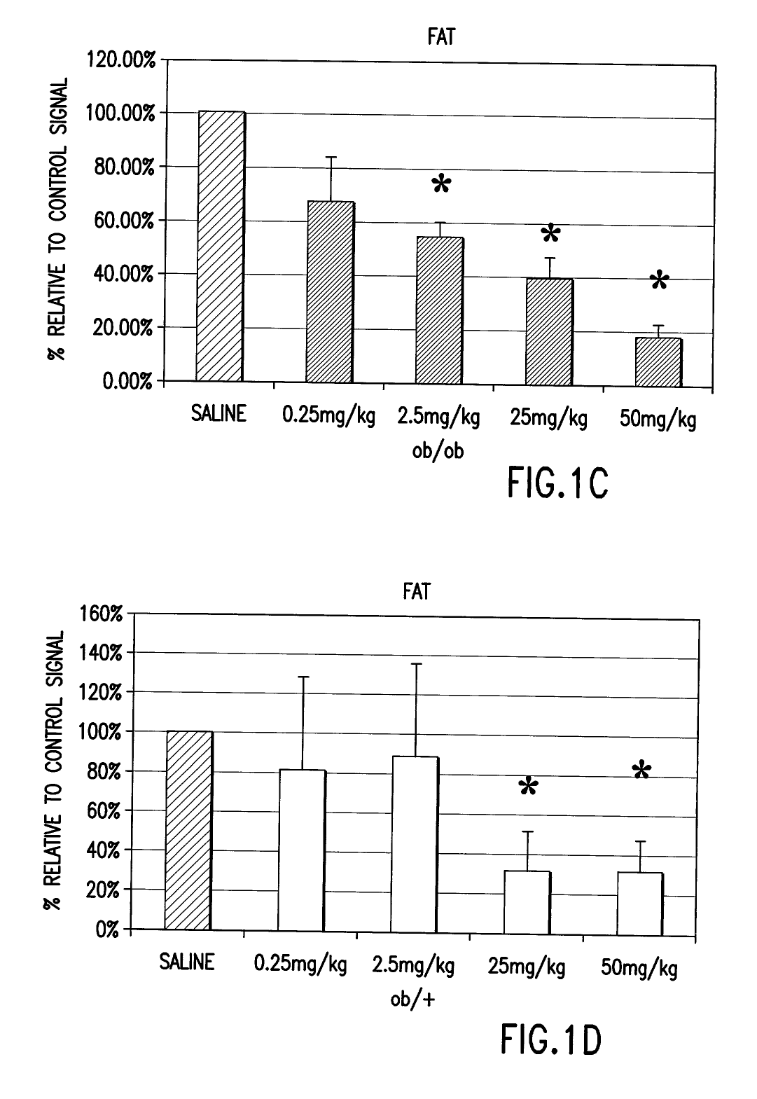

As described detailed in the previous Examples, the inventors undertook a study wherein they developed anti-PTP1B 2′-O-(2-methoxy)-ethyl-modified phosphorothiorate oligonucleotides as described in Example 1. Insulin resistant, hyperglycemic obese (ob/ob) diabetic mice and lean (ob/+) littermates were treated with PTP1B antisense ISIS-113715 (having the nucleotide sequence GCTCCTTCCACTGATCCTGC (SEQ. ID NO: 1) at 50 mg/kg two times per week for 3 weeks or with 25, 2.5 or 0.25 mg/kg two times per week for 6 weeks as described in Example 2. At the completion of the study, liver, fat and muscle were obtained, homogenized and analyzed by immunoblotting as described in Example 3. PTP1B protein expression was decreased in a dose-dependent manner in liver and fat from both ob/ob and ob/+ animals (See FIG. 1). No decrease in PTP1B protein was detected in skeletal muscle.

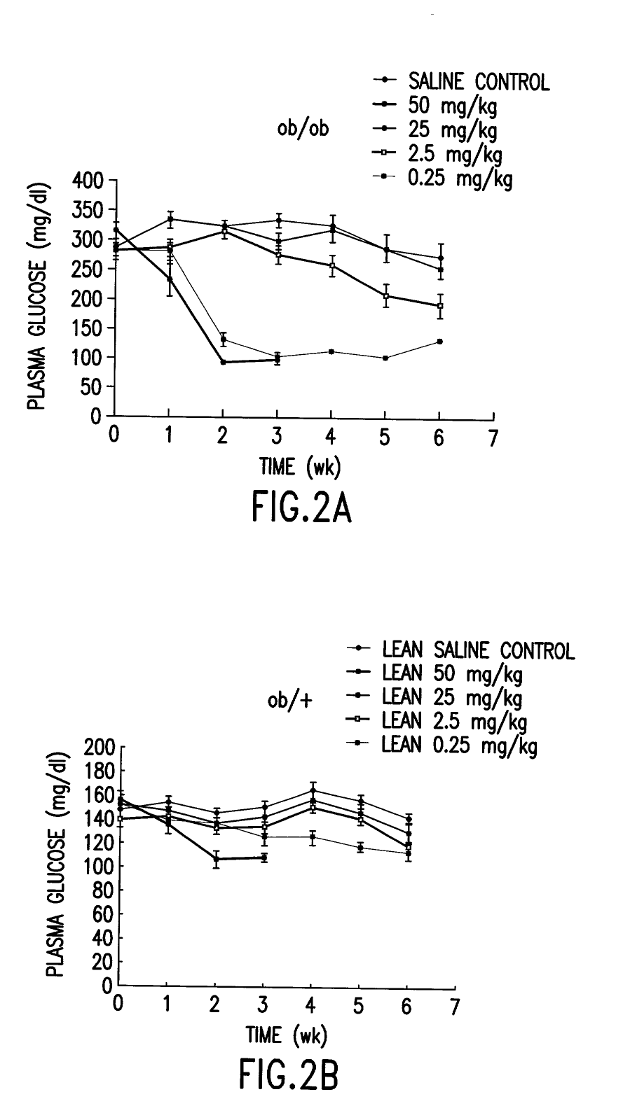

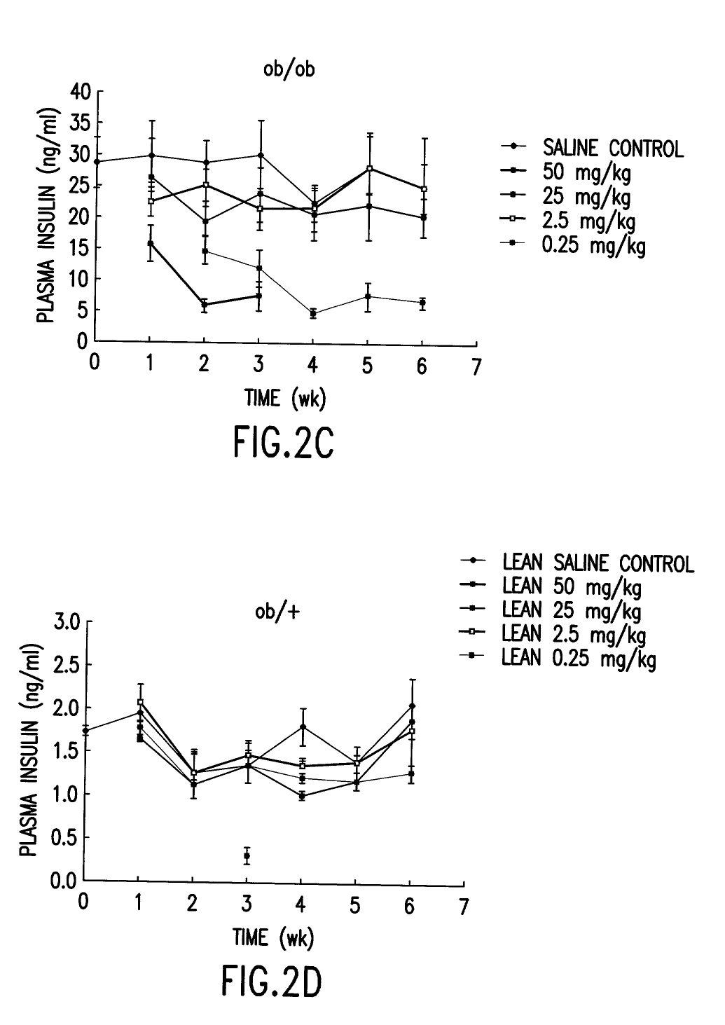

After 6 weeks of treatment in ob/ob mice, total glycated hemoglobin was normalized to lean ob/+ littermate levels. Total glycated hemoglobin was reduced to lean levels from 7.50±0.4% in the saline treated group to 5.30±0.2% (p<0.05) in the 25 mg/kg dose group. Glycated hemoglobin was unchanged in the 2.5 mg/kg group, however, hemoglobin changes occur over 4 to 6 weeks in mice, and the glucose levels were only significantly decreased beginning in week 3. Treatment of diabetic ob/ob mice and their lean (ob/+) littermates with PTP1B antisense oligonucleotides also normalized glucose homeostasis and increased insulin sensitivity (See FIG. 2). PTP1B antisense treatment normalized glucose and improved insulin levels within 2 weeks of treatment (See FIG. 2). PTPIB antisense-treated ob/ob mice dosed i.p. at 2.5 mg/kg or higher concentration of antisense had a significant reduction in blood glucose levels over the duration of treatment with a concomitant decrease in circulating insulin concentrations. Also in PTP1B antisense-treated lean (ob/+) littermates that have impaired glucose tolerance, glucose and insulin levels were reduced compared to saline-treated animals without hypoglycemia. Thus, a decrease in PTP1B protein level in liver and fat correlated with increased insulin sensitivity in both obese and lean animals. Treatment of the animals with a universal control oligonucleotide did not affect PTP1B protein levels or lower glucose and insulin levels in the animals.

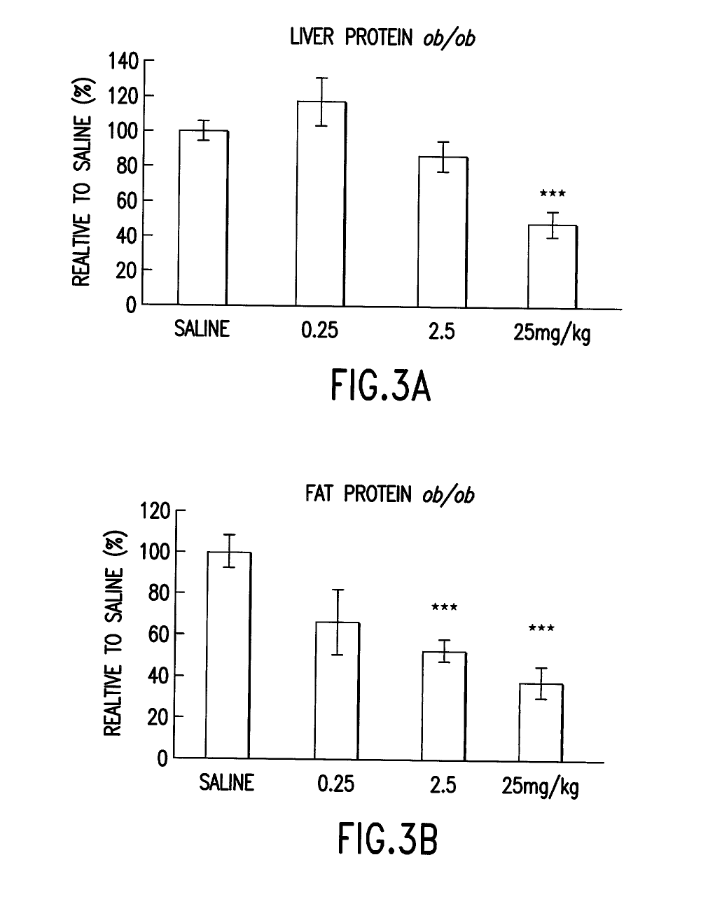

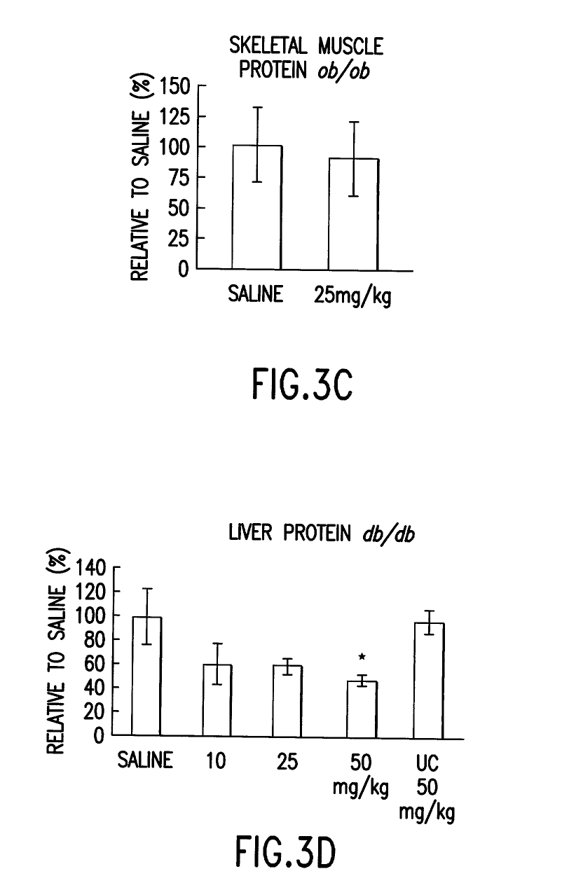

As also described in the Examples, ISIS-113715, developed as described in Example 1, was administered to obese insulin resistant diabetic (ob/ob) and diabetic (db/db) mice. Diabetic ob/ob mice were treated ip. twice per week for six weeks with ISIS-113715 in a dose ranging study described in Example 5. PTP1B protein levels were reduced in liver and fat without a reduction in skeletal muscle (See FIGS. 3A-3C). In the high dose group hepatic mRNA levels were also reduced (4412%, p<0.05). Diabetic db/db mice treated ip. once per week for four weeks in a dose ranging study with ISIS-113715 also had lowered PTP1B protein (See FIG. 3D) and mRNA (55±8%, p<0.05) levels in liver. A Universal Control (UC) combinatorial mixture of oligonucleotides was without effect (FIG. 3D) on PTP1B levels in liver. The UC was synthesized as described in Example 6. The data shown in FIG. 3 are mean±S.E. and statistics were determined as a two tailed t-test with *p<0.05, ***p<0.001.

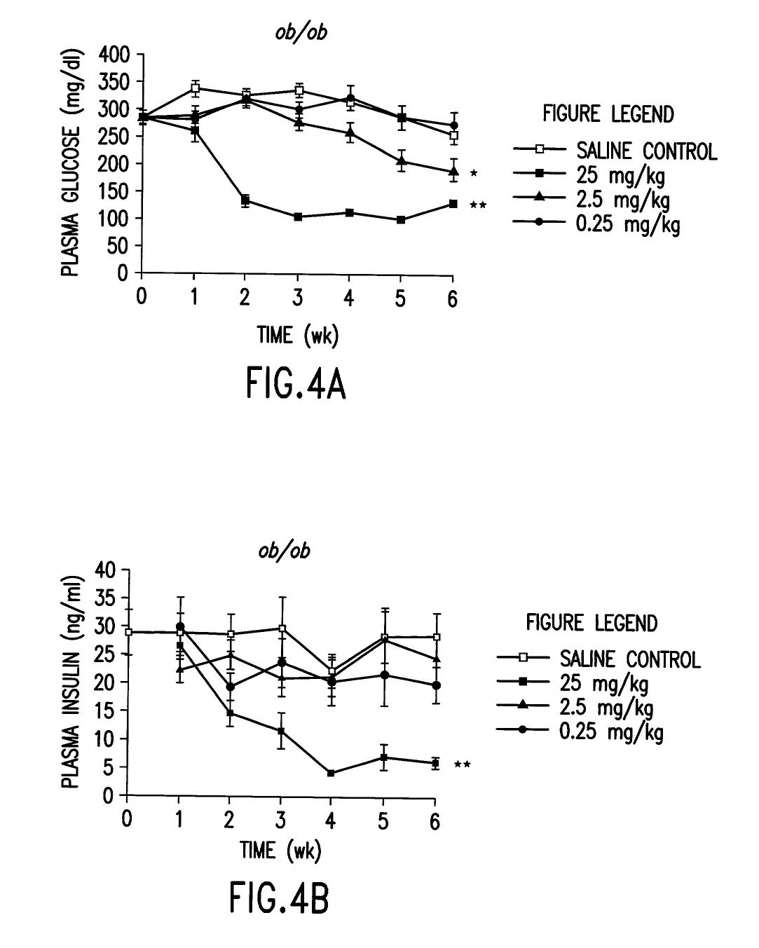

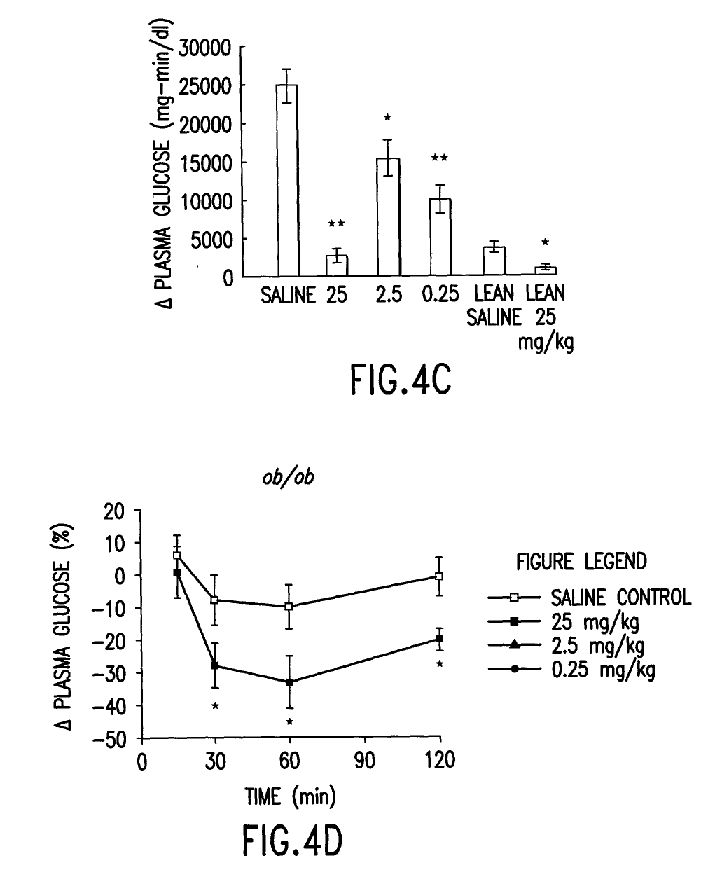

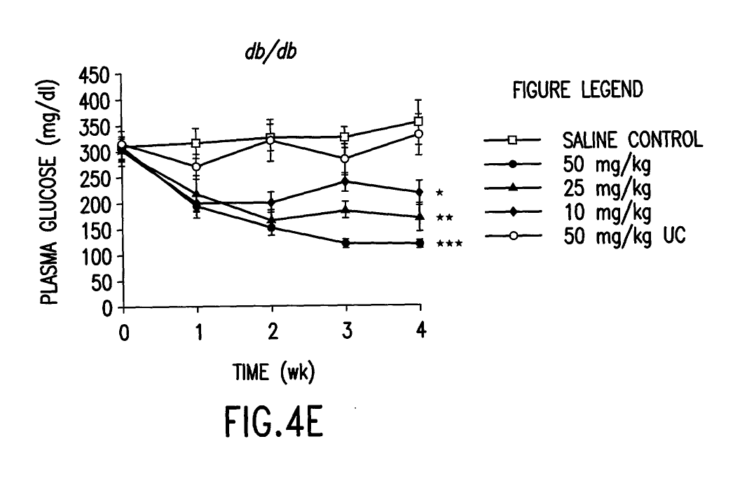

Plasma glucose in ob/ob mice was normalized, after two weeks of treatment, to lean ob/+ levels in the high dose group (25 mg/kg) and was improved in the 2.5 mg/kg dose group by week three (See FIG. 4A). After six weeks of treatment, an overnight fast reduced glucose levels (30%) in the high dose group (139±14 vs. 97±3 mg/dl, p<0.05) with no hypoglycemia. Glycated hemoglobin, HbA_{1C}, a measure of long term glucose homeostasis, was reduced from 6.2±0.3% in saline treated ob/ob mice to 4.7±0.1% (p<0.01) in the high dose group, a level equivalent to lean ob/+ littermates (4.8±0.1%). Plasma insulin levels were decreased 77% (See FIG. 4B) at six weeks in the 25 mg/kg treatment group. Glucose excursion during an i.p. glucose tolerance test (GTT; see Example 7) was normalized in the 25 mg/kg treatment group and improved in the other two dose groups (See FIG. 4C). While ob/+ mice are lean and not diabetic they are insulin resistant and have an impaired glucose tolerance compared to wild type C57BL/6J mice. The PTP1B antisense treated lean ob/+ group also had a statistically significant improved glucose excursion, with no observed hypoglycemia. In ob/ob mice, an enhanced reduction in glucose level (3.4 fold, FIG. 4D) occurred during an insulin tolerance test (ITT) with PTP1B antisense treatment as described in Example 7. The GTT and ITT results suggest enhanced insulin sensitivity that is not due to increased peripheral insulin sensitivity since ISIS-113715 reduces PTP1B protein levels in liver and adipose tissue with no effect in skeletal muscle. In db/db mice glucose levels were dose-dependently improved reaching lean (db/+) littermate levels at 50 mg/kg PTP1B antisense (See FIG. 4E). No change in plasma glucose in db/db mice was observed with 50 mg/kg UC treatment and no effect on glucose level was seen with either ISIS-113715 or UC treatment in db/+ mice. PTP1B antisense treatment with ISIS-113715 was well tolerated in all animals as described in Example 8. Molecular toxicology, blood chemistry, and histological examination indicated that PTP1B antisense treatment at lower doses did not adversely affect liver function or the general health of the ob/ob mice in these studies described in Example 8. The results shown in FIG. 4 are expressed as change from baseline AUC_{Glucose} for GTT (25, 2.5, and 0.25 mg/kg in ob/ob and 25 mg/kg treatments in lean ob/+ littermates). Results are expressed as % change from baseline for ITT. Data are mean±S.E. Statistical evaluation was performed via 1-way ANOVA and t-tests week (See FIG. 3D) liver PTP1B protein level was significantly reduced at the 50 mg/kg dose. The level of significance is given as a two sided test in all studies *p.<0.05, **p<0.01, ***p<0.001. FIG. 4A * weeks 3 through 6, ** weeks 2 through 6; FIG. 4B ** weeks 3 through 6; FIG. 4E*, **, and *** weeks 2 through 4.

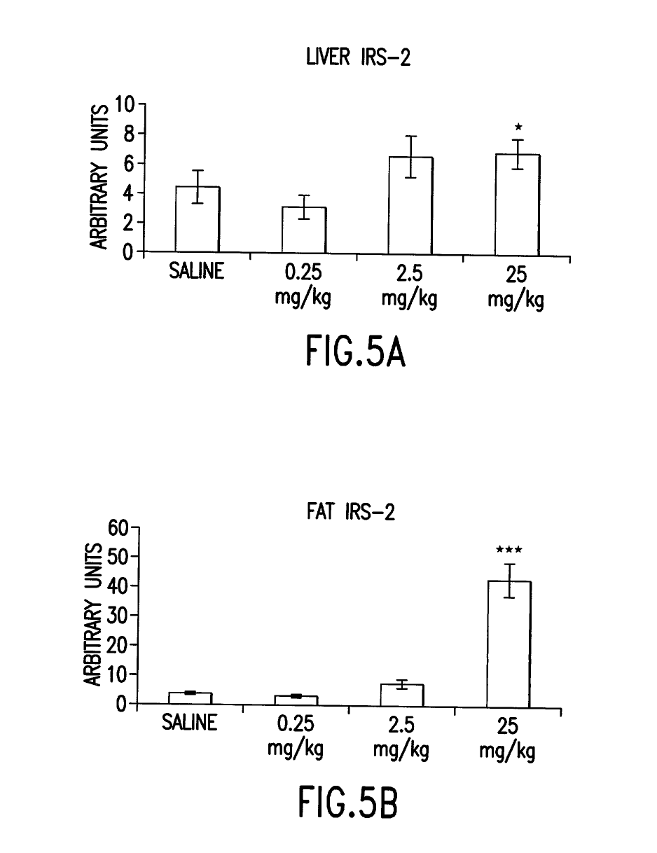

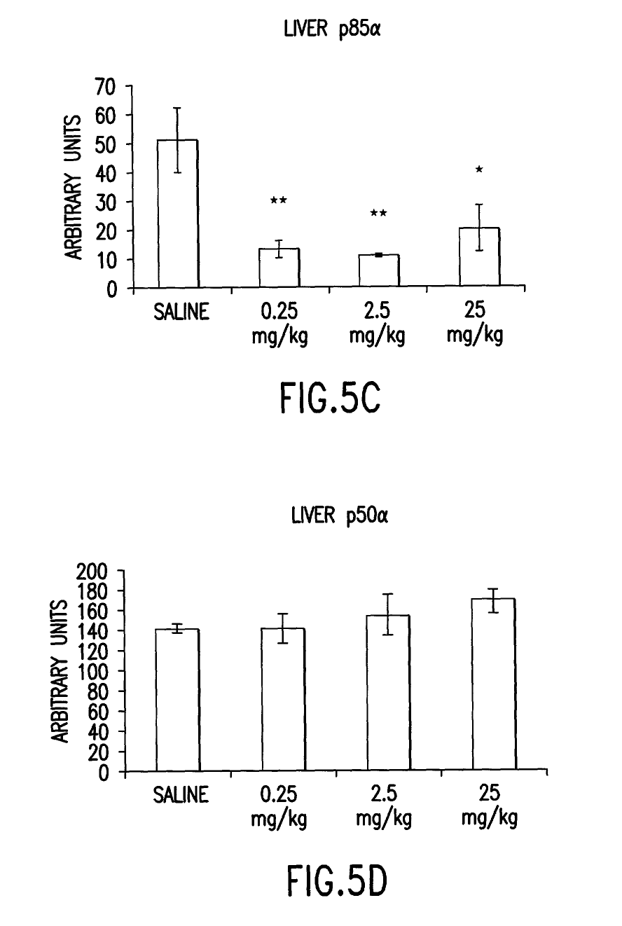

The inventors measured the protein expression of IR, IRS-1, IRS-2, and PI3-kinase isoforms in liver and fat from ob/ob and ob/+ mice treated with ISIS-113715 by immunoblotting with specific antibodies as described in Example 3. No effect in IR or IRS-1 expression were detected in liver and fat from ob/ob or ob/+ mice treated with antisense, although reduced expression of IR, IRS-1 and IRS-2 was confirmed in ob/ob relative to ob/+ mice. Reduced levels of IRS-2, considered important in hepatic insulin signal transduction (Kido, Y., et al., J. Clin. Invest., 105(2):199-205 (2000); Rother, K., et al., J. Bio. Chem., 273(28):17491-7 (1998)), may contribute to hepatic insulin resistance and increased IRS-2 expression and thus could improve hepatic insulin sensitivity. The inventors found that IRS-2 protein levels were increased dose dependently in liver and fat in ob/ob mice treated for 6 weeks with PTP1B antisense (see FIGS. 5A and 5B. The results shown in FIG. 5 are the average of 4 mice within each group. The data are represented as arbitrary units and are the mean±SEM. Statistics were determined as a two tailed t-test with *p<0.05, **p<0.01, and ***p<0.001). Antisense treatment had no effect on IRS-2 levels in the same tissues from lean ob/+ mice.

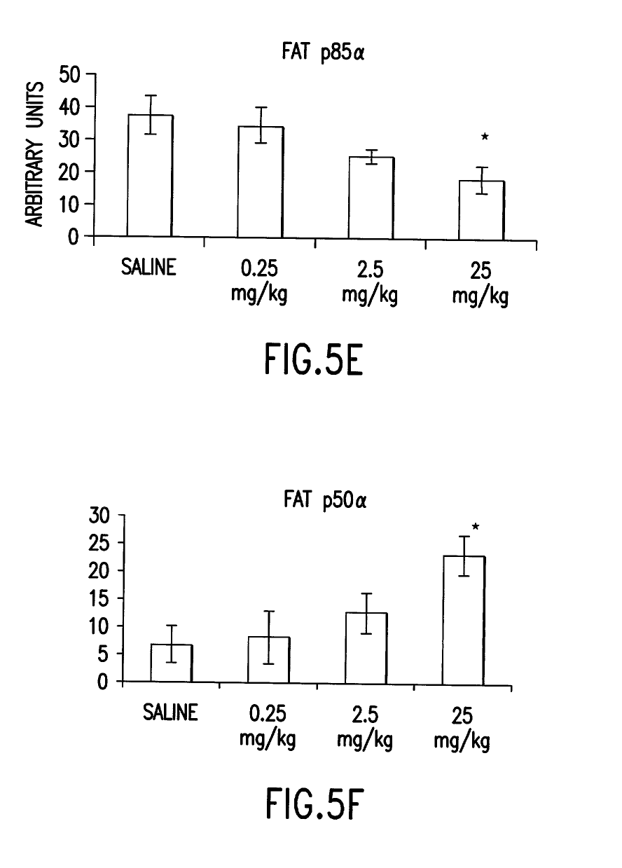

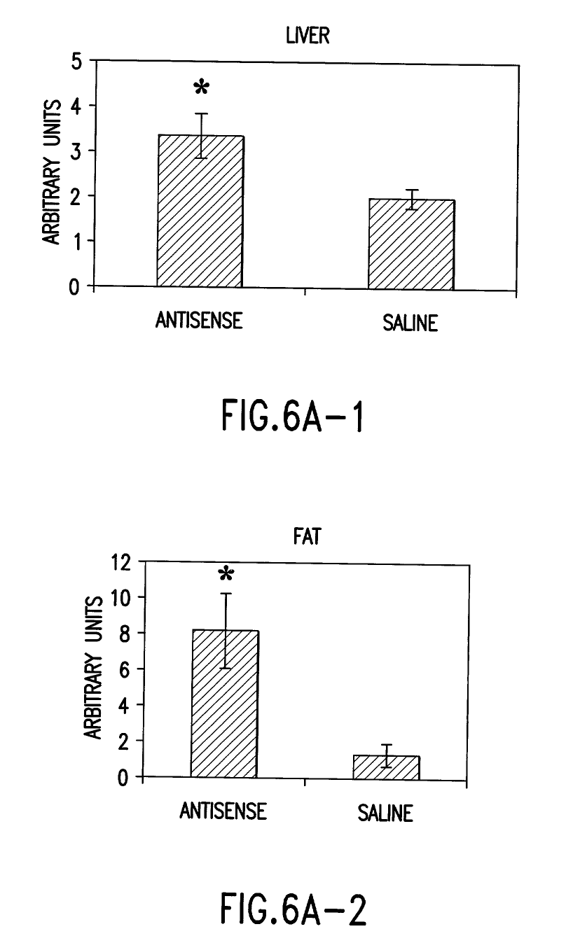

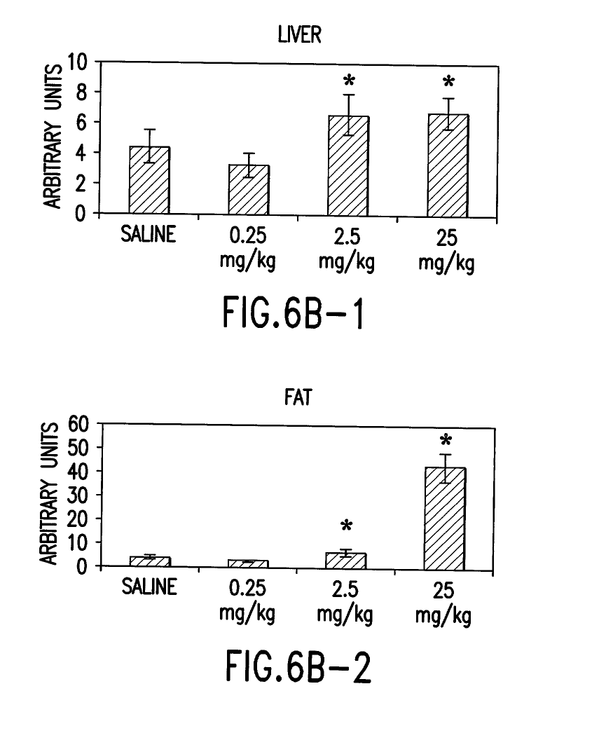

IRS-2 is considered the primary insulin receptor substrate involved in insulin signaling in liver. IRS-2 protein expression is significantly decreased (50-70% in liver) in ob/ob animals vs. ob/+ mice (Kerouz, N. J., et al., J. Clin. Invest., 100(12):3164-72 (1997)). The reduced levels of IRS-2 in liver from ob/ob mice is believed to contribute to hepatic insulin resistance and normalization of IRS-2 expression should restore hepatic insulin sensitivity. Therefore, IRS-2 protein levels were measured in liver and fat obtained from saline control or PTP1B antisense-treated animals (See, FIG. 6). IRS-2 was significally increased 2-fold in liver and 4-fold in fat from ob/ob animals treated with ISIS-113715 50 mg/kg twice a week for 3 weeks (See, FIG. 6A). This effect was increased in a dose-dependent manner (See, FIG. 6B). In contrast, there was no significant change in IRS-2 levels in the tissues from ob/+ mice. Moreover, PTP1B antisense increased the protein expression of IRS-2 only in the obese animals over the basal lean levels.

To determine whether insulin sensitization by treatment with PTP1B antisense was associated with changes in p55% and p50% isoform expression, homogenates of liver and fat from PTP-1B antisense-treated lean and obese animals were immunoblotted with p85 antiserum (See Example 3). A dose dependent reduction of p85α isoform expression in both liver and fat was observed along with increased expression of the p50α isoform in fat (See FIG. 5). In a three week study in ob/ob mice dosed at 50 mg/kg ISIS-113715, a similar phenotype was observed with decreased expression of p85α in liver (40%, p<0.0⁴) and fat (30%, p<0.01). Increased expression of both p50α, 2 fold in liver (p<0.01) and 20 fold (p<0.01) in fat, and p55α, 6 fold (p<0.01) in liver occurred in this study. No changes in PI3-kinase isoform expression were observed in skeletal muscle. The inventors found that differential expression of PI3-kinase regulatory subunits observed in the PTP1B antisense-treated ob/ob mice increased insulin sensitivity in liver and fat and improved glucose tolerance in these diabetic mice.

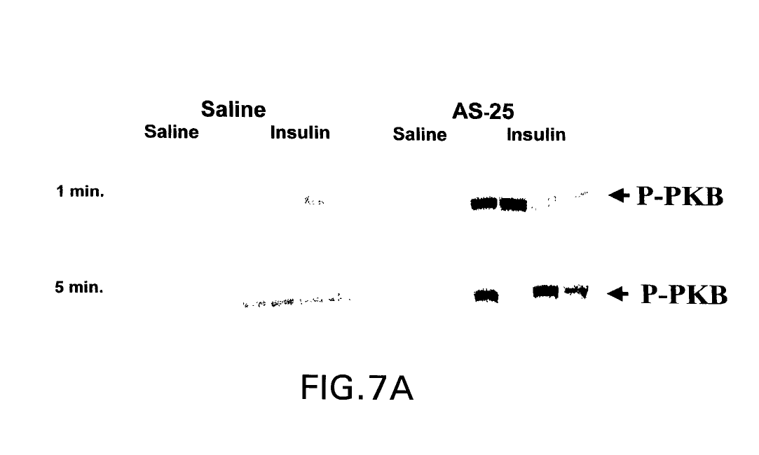

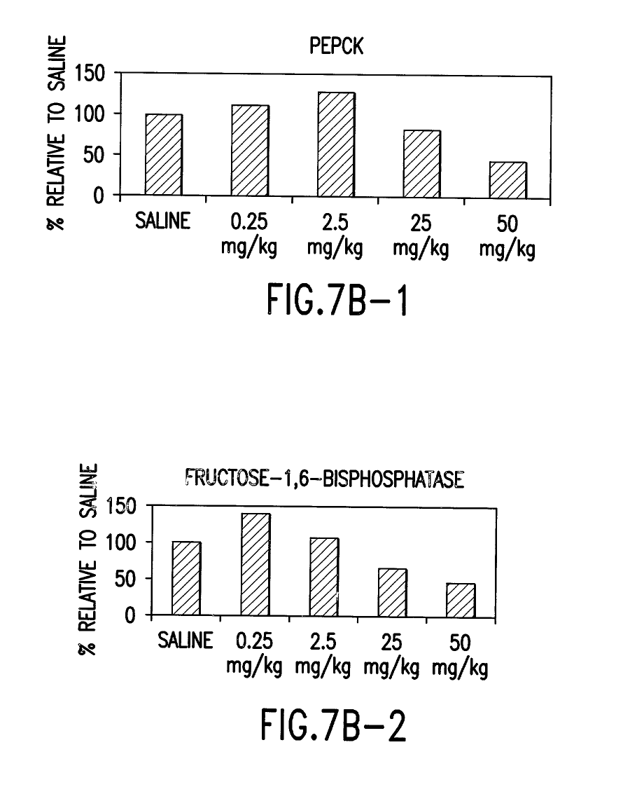

Next, the inventors investigated the effect of the antisense treatment on PKB phosphorylation. An intraperiteneal insulin challenge (2 Units insulin/kg) was performed in ob/ob mice previously treated for six weeks with saline or ISIS-113715 antisense (i.p., 25 mg/kg, twice per week) (Specifically, saline or insulin was administered at 0 minutes after a 5 hours fast. Insulin (or saline control) was given i.p. at 2U/kg in 0.1% BSA (Insulin R, Eli Lilly). Tissue samples for liver (0, 1, 5 minutes), fat (0, 1, 5 minutes) and skeletal muscle (0, 2, 6 minutes) were taken under both saline and insulin stimulated conditions. Within each challenge (saline and insulin) were subgroups of saline or antisense treated (25 mg/kg) mice. Livers were extracted at one or five minutes post insulin challenge. Treatment with ISIS-113715 antisense increased basal, but mainly insulin-induced PKB phosphorylation without changing protein levels (See FIG. 7A). The increased phosphorylation of PKB is evidence of increased insulin sensitivity in the liver of the ISIS-113715-treated ob/ob mice and correlates with the changes in insulin signaling proteins (IRS-2 and PI3-kinase isoform expression) described earlier.

To investigate whether a more efficient insulin signaling cascade in the animals treated with the antisense correlated with a decrease of PEPCK mRNA expression in liver, the inventors measured mRNA levels by Microarray analysis (Affymetrix murine 11K chip) comparing antisense ISIS-1113715-treated animals to saline-treated diabetic ob/ob mice (See Example 4). PTP1B antisense significantly reduced PEPCK mRNA levels (38%) and F-1,6-BP (17%) in ob/ob mice treated for six weeks at 25 mg/kg. In this study a similar reduction was observed for both PEPCK (55%) and F-1,6-BP (52%) mRNA in an independent ob/ob mouse study dosing i.p. twice per week for three weeks with 50 mg/kg ISIS-113715.

In some additional studies, anti-PTP1B 2′-O-(2-methoxy)-ethyl-modified phosphorothiorate oligonucleotides were developed in Example 9. Insulin resistant, hyperglycemic obese (ob/ob) mice were treated with PTP1B antisense I-113715 at 25, 2.5 or 0.25 mg/kg two times per week for 6 weeks. At the completion of the study epididymal fat pads were obtained, homogenized and analyzed by immunoblotting. PTP1B protein expression in epididymal fat pads was decreased by 33%, 46% and 61% by treatment with 0.25, 2.5 and 25 mg/kg PTP1B antisense oligonucleotide respectively.

First, the inventors examined whether PTP1B reduction had an effect on body weight. There was no significant difference on body weight between animals treated with saline and those treated with 0.25 or 2.5 mg/kg over the duration of the study. However, the 25 mg/kg group gained 15% less weight by 5 weeks. (See FIG. 8A; values in this figure are expressed as the mean±SEM (n=10)). Growth rate per week was not different between treatments, but a small but significant difference was found with the highest dose of antisense treatment (25 mg/kg) at 5 weeks (See, FIG. 8B; values in this figure are expressed as the mean±SEM (n=10)).

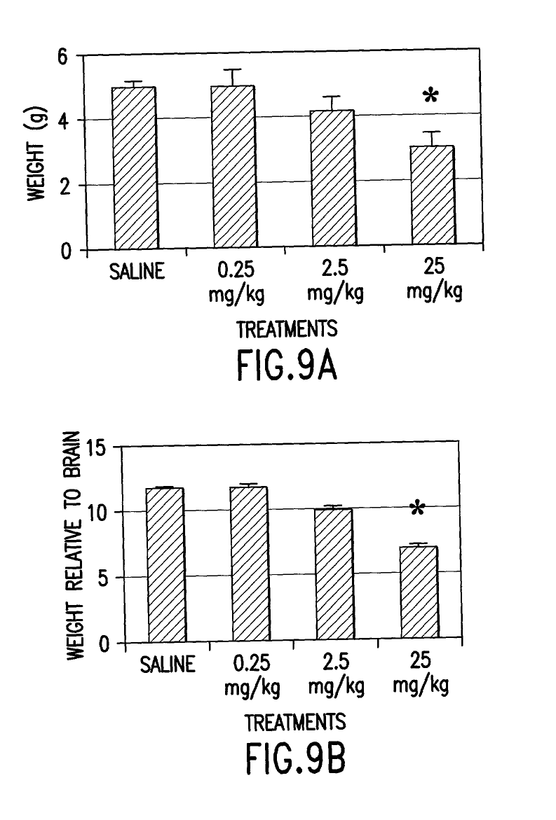

To investigate whether the decrease in body mass in the group of animals treated with the higher concentration of antisense could be the consequence of a reduction on fat stores, epididymal fat pads were obtained and weighted. Epididymal fat weight was unchanged with the lower dose treatment (See FIG. 9; values in this figure are expressed as the mean±SEM (n=10)*p<0.05 as compared with ob/ob mice treated with saline) and a minimal effect was detected with 2.5 mg/kg of ASO. In contrast, fat weight was reduced by 42% in animals treated with 25 mg/kg ASO (P<0.05, normalized to brain weight) in correlation with the reduction in body weights.

As PTP1B antisense treated mice showed a decrease of adiposity, the inventors investigated whether a molecular mechanism for reducing adipose tissue mass existed. To investigate whether genes coding proteins involved in lipid metabolism were altered by PTP1B reduction, an integrated gene expression study using fat samples was performed. Total RNA was isolated from epididymal fat from ob/ob mice treated with or without 25 mg/kg PTP1B antisense oligonucleotide and analyzed by murine 11K oligonucleotide microarray (Affymetrix, Santa Clara, Calif.) or hybridized to the mouse Gem 2 microarray (Incyte Pharmaceuticals, Palo Alto, Calif.), as described in Example 9. PTP1B antisense resulted in a coordinate decrease in the expression of genes involved in fatty acid homeostasis as shown below in Table 1.

TABLE 1

Fold Change

| | Oligonucleotide | Gem 2 |
|---|---|---|
| Gene | microarray | microarray |
| Lipid Metabolism | | |
| SteroylCoA desaturase (scd1) | −1.7 | −3.65 |
| StearoylCoA desaturase (scd2) | −3.4 | −2.5 |
| Glutathione S transferase | −3.2 | −3.6 |
| Fatty Acid Synthase | −2.5 | −3.9 |
| glycerol3-Pacyltransferase | −2.1 | −2.1 |
| LPL | −2.3 | ND |
| ATP citrate-lyase | −1.8 | ND |
| Pyruvate decarboxylase | −2.1 | −3.1 |
| Spot14 | −8.2 | −6.6 |
| HSL | −2.2 | −3.65 |
| Transcription factors | −2.7 | ND |
| ADD1/SREBP | | |
| Adipocyte-specific genes | −2.5 | −2.1 |
| PPARgamma | | |

The fold changes were determined by microarray hybridization (n = 3) pools from 4 mice
ND, non-detectable or low signal-to noise ratio

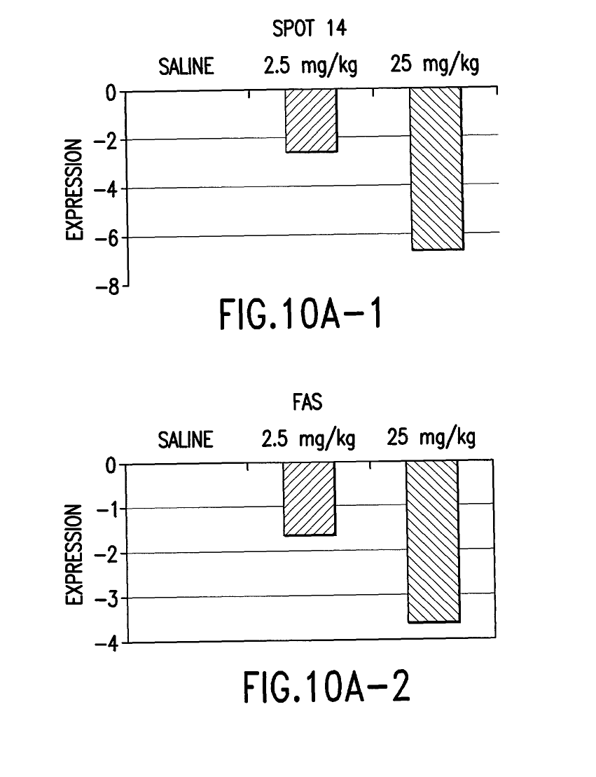

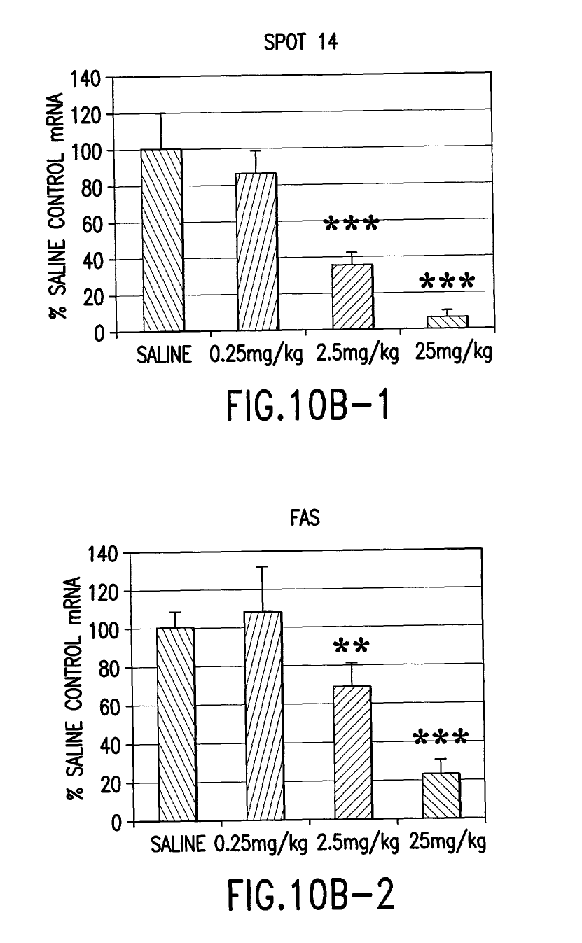

Interestingly, PTP1B antisense treatment decreased expression of the transcription factor SREBP1 which regulates several genes involved in lipogenesis in mature adipocytes (Osborne, et al., J. Biol. Chem. 275:32379-32382 (2000)). The inventors also found downregulation of expression of several SREBP1 target genes involved in fatty acid synthesis including spot14, ATP citrate-lyase, fatty acid synthase, SteroylCoa desaturases, as well as lipoprotein lipase and PPARλ, a member of the nuclear hormone receptor family of ligand—activated transcription factors that plays a pivotal role in fat cell differentiation (See Table 1 above). The gene expression changes of some of these genes were confirmed by hybridization to the Mouse Gem 2 microarray (See Table 1) and using quantitative PCR (hereinafter “qPCR”). FIG. 10A showed a dose-response reduction in Spot 14 and Fatty acid synthase gene expression by microarray that correlated with a dose-response reduction of Spot14 and FAS measured by qPCR analysis (See FIG. 10B; the values expressed in FIG. 10A and 10B are expressed as the mean±SEM (n=4) **p<0.01 and ***p<0.001 as compared with ob/ob mice treated with saline).

More specifically, FIG. 10A shows the results of total RNA isolated from epididymal fat pads of ob/ob mice treated with different concentrations of PTP1B antisense oligonucleotides and analyzed by murine 11K oligonucleotide microarray (Affymetrix, Santa Clara, Calif.). The expression level of FAS and Spot14 in ob/ob mice treated with 2.5 or 25 mg/kg PTP1B antisense were compared with that in ob/ob mice treated with saline and fold change of expression in antisense treated vs. saline treated mice calculated using Affymetrix Gene Chip 3.2 software. FIG. 10B shows the results of total RNA isolated from epididymal fat pads of ob/ob mice treated with different concentrations of PTP1B antisense oligonucleotides and analyzed by qPCR as described in Example 9. Spot14 and FAS are critical genes involved in fatty acid synthesis from glucose.

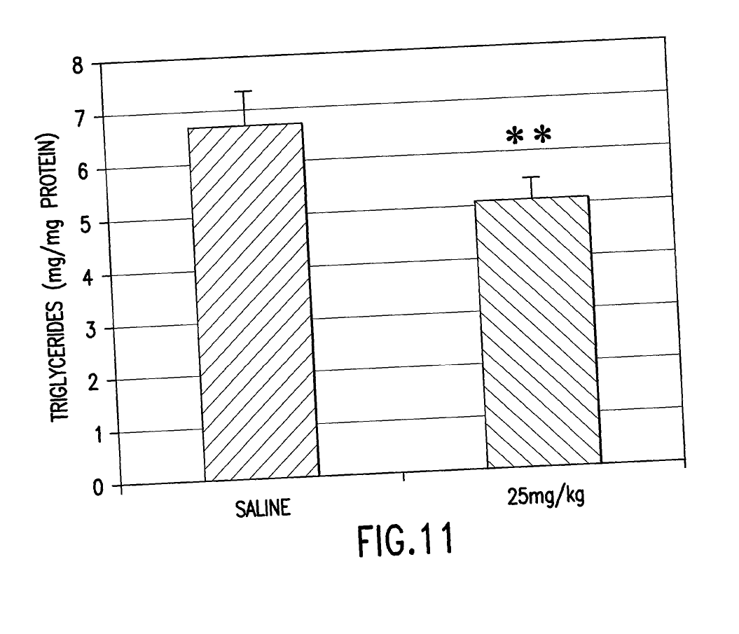

The triglyceride content of fat from PTP1B antisense treated mice was significantly decreased, confirming the physiological significance of a decreased SREBP1c expression in these mice (See FIG. 11; the values in this figure are expressed as the mean±SEM (n=4) *p,0.01 compared to saline treated controls).

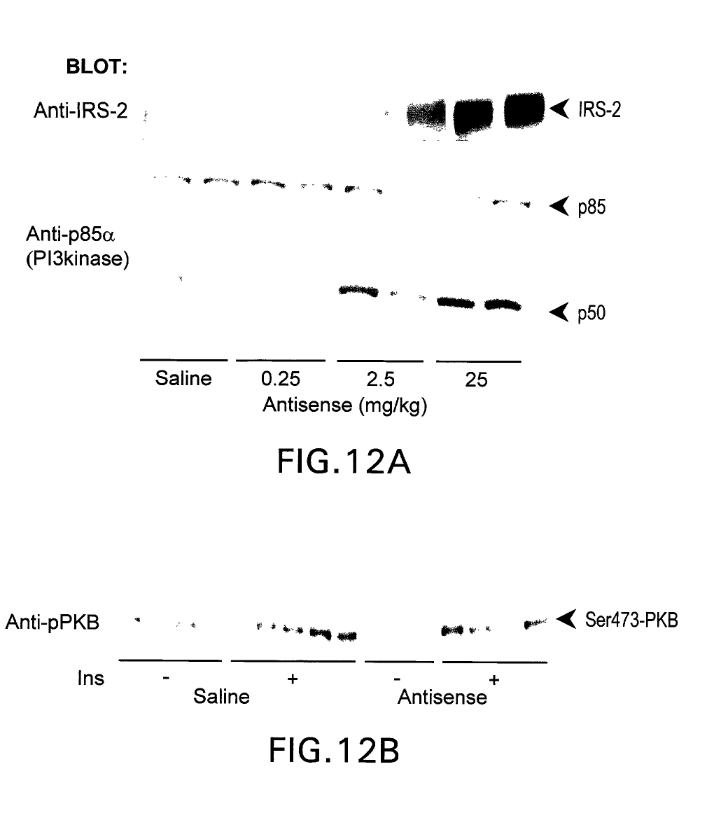

The inventors measured the protein expression of IR, IRS-1, IRS-2, and PI3-kinase isoforms in fat by immunoblotting with specific antibodies. No significative differences in IR or IRS-1 expression were detected in fat from obese animals treated with antisense. In contrast, IRS-2 significantly increased in a dose response manner in fat from ob/ob animals treated with I-113715 (See FIG. 12). In addition, a change in expression level of splice variants of p85α in fat (See FIG. 12) was observed in PTP1B antisense treated animals. This change was characterized by a reduction in p85α and an up-regulation of the p50α isoform.

To investigate whether an increase in IRS-2 expression and/or differential expression of PI3-kinase regulatory subunits observed in fat from the PTP1B antisense-treated ob/ob mice would increase insulin sensitivity, an intraperitoneal insulin challenge (2 Units insulin/kg) was performed in ob/ob mice previously treated for six weeks with saline or I-113715 antisense (i.p., 25 mg/kg, twice per week). Fat was extracted at one minute post insulin challenge and general tyrosine phosphorylation of proteins and PKB phosphorylation at Ser 473 was measured. Treatment with I-113715 antisense had no effect on tyrosine phosphorylation of the insulin receptor nor on basal, or insulin-induced PKB phosphorylation (See FIG. 12).

More specifically, FIG. 12A shows lysates obtained obtained from epididymal fat pads extracted from ob/ob mice treated with different concentrations of PTP1B antisense oligonucleotide for 6 weeks were separated in a 7.5% SDS-PAGE, transferred and immunoblotted using anti-IRS-2 and anti-p85 antibodies as described in Example 9. FIG. 12B shows that antisense (25 mg/kg for 6 weeks) or saline treated ob/ob mice were fasted for 5 hours and then challenged with an i.p. bolus of saline or insulin (2U/kg in 0.1% BSA). Epididymal fat was taken at 1 min following the challenge and lysates were prepared and separated in a 10% SDS-PAGE, transferred and immunoblotted using anti-phosphoserine 473-PKB antibodies.

In the two separate experiments described in Example 10, ob/ob mice were treated with ISIS-113715 for 3 weeks at a dose of 50 mg/kg twice per week or for 6 weeks in a dose response study at 25, 2.5 and 0.25 mg/kg twice per week. Reduction of PTP1B mRNA was seen at 50 and 25 mg/kg treatment levels in liver, and PTP1B protein levels were reduced in all treatment groups in liver and fat with no change in expression in muscle (See Table II below).

TABLE II

| | PTP1B | PTP1B | PTP1B | | | |
|---|---|---|---|---|---|---|
| | RNA % | Protein % | Protein % | | | Epidymal |
| | Reduction | Reduction | Reduction | | | Fat |
| Treatment | (Liver) | (Liver) | (Fat) | Glucose | Insulin | Weight |
| 50 mg/kg | 52% | 60% | 84% | Normalized | 78% | NR |
| (3 wk) | | | | | | |
| 25 mg/kg | 53% | 52% | 61% | Normalized | 77% | 42% |
| (6 wk) | | | | | | |
| 2.5 mg/kg | 16% | 14% | 46% | 30% | 13% | 17% |
| (6 wk) | | | | Decrease | | |

RNA and protein levels are shown as percent reduction compared to vehicle-treated control. Insulin is shown as percent reduction compared to before treatment. For insulin values, a reduction of 95.5% would be normalized. The fat weight is normalized to brain weight. NR-Not Recorded. Plasma glucose levels were normalized to lean (ob/+) levels in the 50 and 25 mg/kg treatment groups, and plasma glucose levels were improved in the 2.5 mg/kg treatment group. Plasma insulin levels were greatly decreased in the 50 and 25 mg/kg treatment group (78.7 and 77%, respectively). In addition, an enhanced reduction in glucose level occurred during an insulin tolerance test with ASO treatment. Epididymal fat weight was reduced 42% and 17% in animals treated at 25 and 2.5 mg/kg, respectively, compared to saline-treated controls.

RNA was harvested from white adipose tissue (WAT) from saline or PTP1B ASO-treated mice. The integrity of the RNA was confirmed using an Agilent 2100. RNA was pooled from the different treatment groups and hybridized to the Affymetrix MG-U74 v2 microarray chip. The results from the 25 mg/kg treatment group were repeated for confirmation. FIG. 13 shows some of the gene expression changes seen with six weeks of PTP1B ASO treatment at 25 and 2.5 mg/kg. In addition, gene expression changes seen with PTP1B ASO treatment for three weeks at 50 mg/kg for three weeks are shown in a separate column. Many of the genes that change in expression were shown to be differentially expressed in adipose tissue between ob/ob and lean mice such as Spot 14, adipsin, retinol-binding protein and ATP citrate lyase (See Nadler, et al., PNAS, 97:11371-11376 (2000)). These genes have also been shown to be upregulated during adipocyte differentiation (Corenelius, et al., Annu. Rev. Nutr., 14:99-129 (1994)). In fact, the microarray results show that approximately half of the genes shown to be regulated during adipocyte differentiation were regulated in the opposite manner with PTP1B ASO treatment. This is in contrast to treatment with TZDs, which have been shown to act by causing adipocyte differentiation and have been shown to upregulate genes indicative of mature adipocytes (Kletzien, et al., Mol. Pharmacol., 41:393-398 (1992); Hallakou, et al., Diabetes, 46:1393-1399 (1997)). In fact, genes such as adipsin, c-Cbl-associating protein and plasminogen activator inhibitor-1 (PAI-1), which are expressed in highly differentiated adipocytes, have been shown to be upregulated by treatment with rosiglitazone or other TZDs and are downregulated with treatment with ISIS-113715 (See FIG. 13) (Ihara, et al., FASEB J., 15:1233-1235 (2001); Baumann, et al., J. Biol. Chem., 275:9131-9135 (2000); Okazaki, et al., Endocr. J., 46:795-801 (1999)).

Table III below shows a list of genes that have been shown to be upregulated with TZD treatment in adipocytes and were downregulated with PTP1B ASO treatment.

TABLE III

| Gene Name | PTP1B ASO | TZD | Reference |
|---|---|---|---|
| c-Cbl-associated protein (CAP) | Decrease | Increase | Ribon¹ |
| ATP citrate lyase mRNA | Decrease | Increase | Way² |
| Glycerol-3-phosphate | Decrease | Increase | Way² |
| dehydrogenase | | | |
| Lactate dehydrogenase-B | Decrease | Increase | Way² |
| Stearoyl-CoA desaturase | Decrease | Increase | Way² |
| Pyruvate carboxylase | Decrease | Increase | Way² |
| Phospjoenolpyruvate | Decrease | Increase | Way² |
| carboxykinase | | | |
| Lipoprotein Lipase | Decrease | Increase | Way² |
| Malic enzyme | Decrease | Increase | Way² |
| Long chain fatty acyl-CoA | Decrease | Increase | Way² |
| synthetase | | | |
| Glycerol-3-phosphate | Decrease | Increase | Way² |
| acetyltransferase | | | |
| Adipsin | Decrease | Increase | Okazaki³ |
| AdipoQ (adiponectin) | Decrease | Increase | Maida⁴ |
| PAI-1 (plasminogen activator | Decrease | Increase | Ihara⁵ |
| inhibitor) | | | |
| PPAR gamma | Decrease | Increase | Spiegelman⁶ |
| StearoylCoA desaturase(scd2) | Decrease | Increase | Way² |
| Fatty Acid Synthase | Decrease | Increase | Way² |
| Stearoyl-coenzyme A desaturase 1 | Decrease | Increase | Way² |

¹Ribon, et al., Proc. Natl. Acad. Sci. USA 95: 14751-14756 (1998).
²Way et al., Endocrinology, 142: 1269-1277 (2001).
³Okazaki et al., Endocr. J., 46: 795-801 (1999).
⁴Maida et al., Diabetes, 50: 2094-2099 (2001).
⁵Ihara et al., FASEB J., 15: 1233-1235 (2001).
⁶Spiegelman et al., Diabetes, 47: 507-514 (1998).

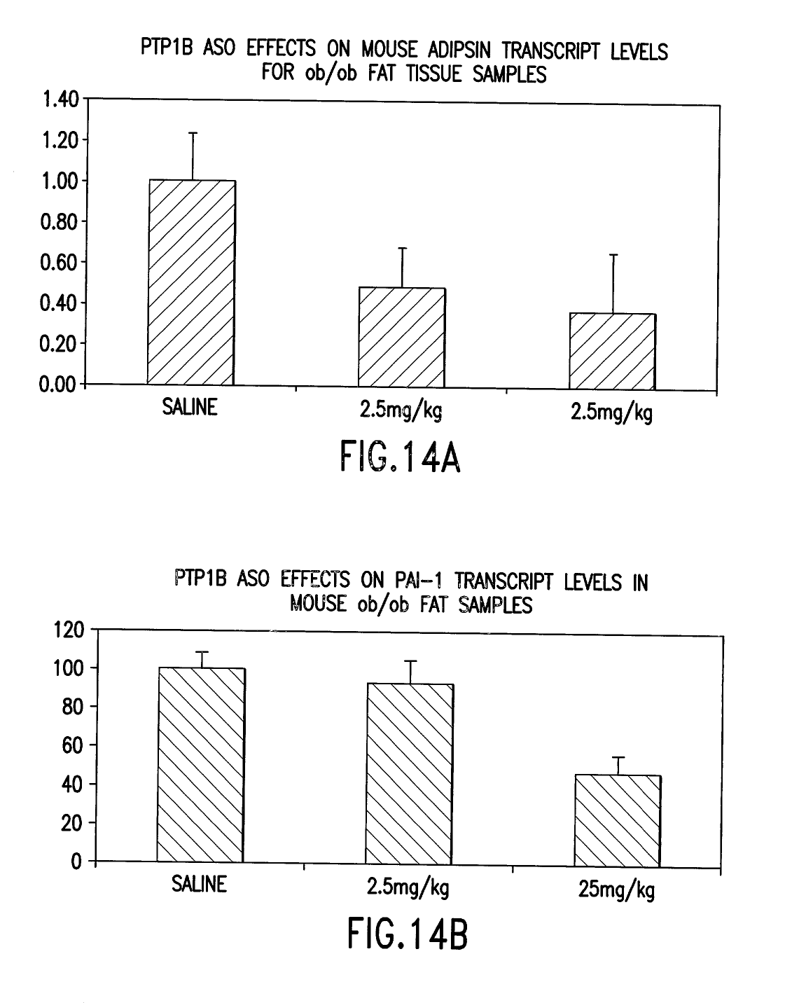

The gene expression changes of some of these genes were confirmed with quantitative PCR. FIG. 14A and B shows the gene expression changes for PAI-1 and adipsin. The results show good correlation between the microarray results and the Q-PCR analysis. Q-PCR was also done on Spot14 and fatty acid synthase, and the microarray results for these genes were confirmed as well.

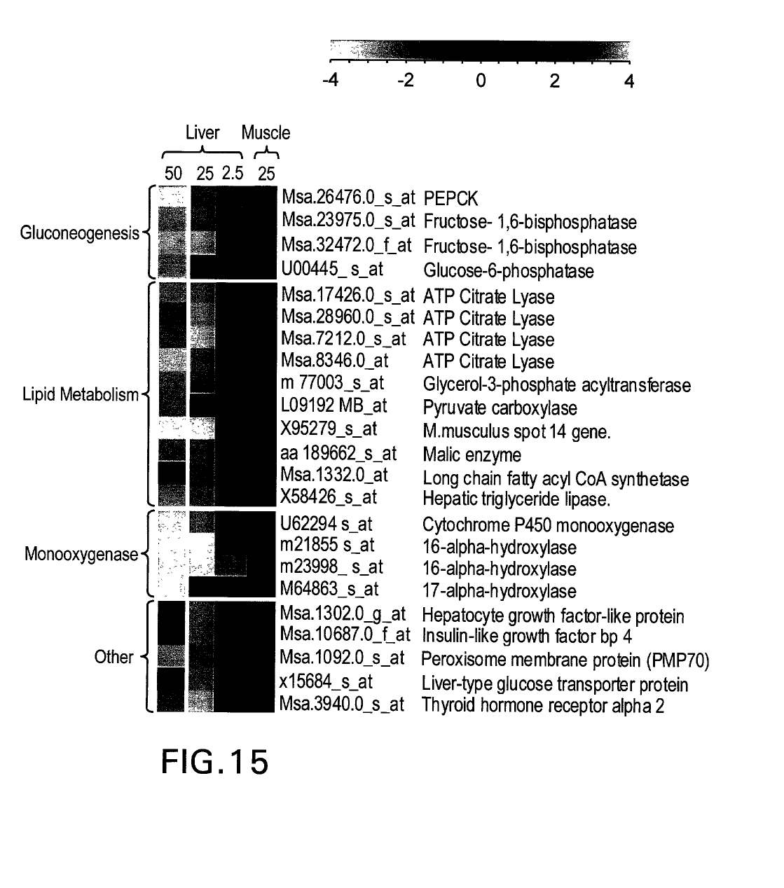

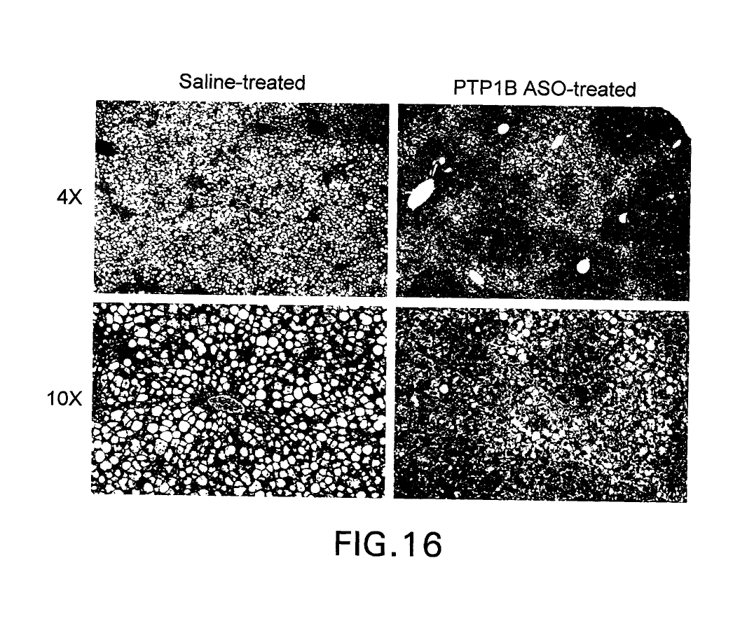

FIG. 15 shows a heat map of gene expression changes in the liver and muscle from ob/ob mice treated with PTP1B ASO compared to saline-treated controls. The results show that several genes that are involved in lipogenesis such as ATP-citrate lyase, Spot-14 and pyruvate carboxylase are downregulated in the liver with PTP1B ASO treatment. In agreement with this, histopathology findings showed reduced levels of lipid in livers from ob/ob mice treated with PTPlB ASO. FIG. 16 depicts the difference in histologically detectable hepatocellular lipid accumulationin the livers of ob/ob mice treated with PTP1B ASO compared to saline control mice. Saline treated mice consistently exhibited marked diffuse hepatocellular lipid accumulation whereas PTP1B ASO treated mice exhibited mild, or occasionally moderate focal to multifocal hepatocellular lipid accumulation.

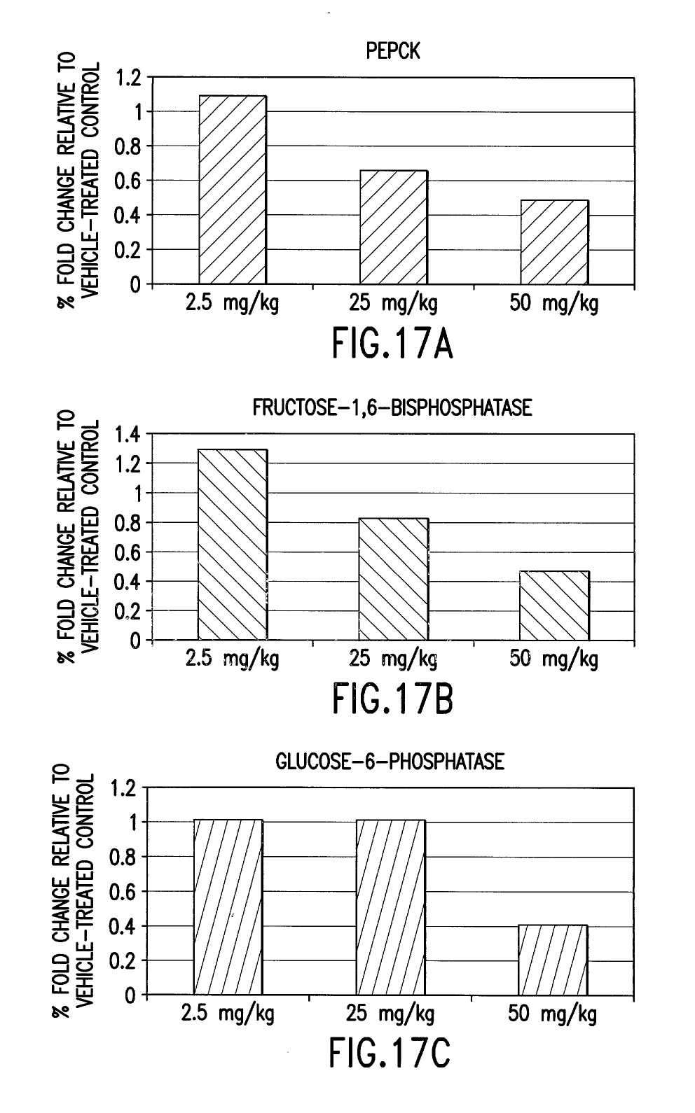

FIG. 17 shows the gene expression changes for phosphoenolpyruvate carboxykinase (PEPCK), fructose-1,6-bisphosphatase, and glucose 6-phosphatase in livers of ob/ob mice treated with PTP1B ASO. The expression of these genes is downregulated with high-dose treatments of PTP1B ASO.

All references, patents and patent applications referred to herein are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. A method for identifying a test compound which inhibits protein tyrosine phosphatase 1B (PTP1B) expression in liver or fat of a non-human mammal, the method comprising the steps of:
(a) treating an insulin resistant non-human mammal with said test compound for a time and under conditions sufficient to allow for a change in the level of expression of at least one of PTP1B mRNA or protein in the liver or fat of said mammal;
(b) removing the fat or liver from said mammal;
(c) detecting the levels of phosphotidylinositol-3-kinase p85α and p50α and/or p55α isoforms in said liver or fat; and
(d) determining whether the test compound inhibits at least one of PTP 1 B mRNA or protein in said liver or fat based upon on the levels of p85α, p50α and/or p55α detected in said liver or fat.

2. The method of claim 1 wherein the non-human mammal is a mouse, rat, monkey, chimpanzee, dog or cow.

3. The method of claim 1 wherein the test compound a protein, oligopeptide, organic molecule, polysaccharide, oligonucleotide or polynucleotide.

4. The method of claim 3 wherein the test compound is an antisense oligonucleotide or antisense polynucleotide.

5. A method for identifying a test compound which downregulates protein tyrosine phosphatase 1B (PTP1B) expression in liver or fat of a non-human mammal, the method comprising the steps of:
(a) treating an insulin resistant non-human mammal with said test compound for a time and under conditions sufficient to allow for a change in the level of expression of at least one of PTP1B mRNA or protein in the liver or fat of said mammal;
(b) removing the fat or liver from said mammal;
(c) detecting the levels of phosphotidylinositol-3-kinase p85α and p50α and/or p55α isoforms in said liver or fat; and
(d) determining whether the test compound downregulates the level of expression of at least one of PTP1B mRNA or protein in said liver or fat based upon on the levels of p85α, p50α and/or p55α detected in said liver or fat.

6. The method of claim 5 wherein the non-human mammal is a mouse, rat, monkey, chimpanzee, dog or cow.

7. The method of claim 5 wherein the test compound a protein, oligopeptide, organic molecule, polysaccharide, oligonucleotide or polynucleotide.

8. The method of claim 7 wherein the test compound is an antisense oligonucleotide or antisense polynucleotide.

9. A method for identifying a test compound which increases insulin sensitivity and reduces blood glucose in an insulin resistant non-human mammal, the method comprising the steps of:
(a) treating an insulin resistant non-human mammal with a test compound for a time and under conditions sufficient to allow for reduced level of expression of at least one of PTP1B mRNA or protein in the liver or fat of a non-human mammal;
(b) removing the fat or liver from said mammal;
(c) detecting the levels of phosphotidylinositol-3-kinase p85α and p50α and/or p55α isoforms in said liver or fat of said non-human mammal; and
(d) determining whether said test compound increases insulin sensitivity and reduces blood glucose in said non-human mammal based upon an the levels of p85α, p50α and/or p55α detected in said liver or fat of said non-human mammal.

10. The method of claim 9 wherein the non-human mammal is a mouse, rat, monkey, chimpanzee, dog or cow.

11. The method of claim 9 wherein the test compound a protein, oligopeptide, organic molecule, polysaccharide, oligonucleotide or polynucleotide.

12. The method of claim 11 wherein the test compound is an antisense oligonucleotide or antisense polynucleotide.

13. A method for identifying a composition which increase the levels of IRS-2 in the liver of an insulin resistant, obese non-human mammal, the method comprising the steps of:
(a) treating an insulin resistant non-human mammal with a composition for a time and under conditions sufficient to allow for a change in the level of expression of IRS-2 in the liver of a nonhuman mammal;
(b) removing the liver of said mammal;
(c) detecting the levels of IRS-2 in said liver of said non-human mammal; and
(d) determining whether said composition increases the level of IRS-2 in the liver of said non-human mammal.

14. The method of claim 13 wherein the non-human mammal is a mouse, rat, monkey, chimpanzee, dog or cow.

15. The method of claim 14 wherein the test compound a protein, oligopeptide, organic molecule, polysaccharide, oligonucleotide or polynucleotide.

16. The method of claim 13 wherein the test compound is an antisense oligonucleotide or antisense polynucleotide.

17. A method for identifying a test compound which downregulates the level of expression of at least one gene involved in lipogenesis, the method comprising the steps of:
(a) treating an insulin resistant non-human mammal with a test compound for a time and under conditions sufficient to allow for a reduction in the level of expression of at least one gene involved in lipogenesis a non-human mammal;
(b) removing the fat from said mammal;
(c) detecting the level of expression of at least one gene involved in lipogenesis in said fat of said non-human mammal; and
(d) determining whether said test compound downregulates the level of expression at least one gene involved in lipogenesis in said non-human mammal.

18. The method of claim 17 wherein the non-human mammal is a mouse, rat, monkey, chimpanzee, dog or cow.

19. The method of claim 17 wherein the test compound a protein, oligopeptide, organic molecule, polysaccharide, oligonucleotide or polynucleotide.

20. The method of claim 19 wherein the test compound is an antisense oligonucleotide or antisense polynucleotide.

21. A method for identifying a test compound which downregulates the level of expression of at least one gene involved in gluconeogenesis, the method comprising the steps of:
(a) treating an insulin resistant non-human mammal with a test compound for a time and under conditions sufficient to allow for a reduction in the level of expression of at least one gene involved in gluconeogenesis a non-human mammal;
(b) removing the fat from said mammal;
(c) detecting the level of expression of at least one gene involved in gluconeogenesis in said fat of said non-human mammal; and
(d) determining whether said test compound downregulates the level of expression of at least one gene involved in gluconeogenesis in said non-human mammal.

22. The method of claim 21 wherein the non-human mammal is a mouse, rat, monkey, chimpanzee, dog or cow.

23. The method of claim 21 wherein the test compound a protein, oligopeptide, organic molecule, polysaccharide, oligonucleotide or polynucleotide.

24. The method of claim 23 wherein the test compound is an antisense oligonucleotide or antisense polynucleotide.
